SEQUENCE LISTING (40 sequences)

SEQ ID NO: 1 - protein, 130 aa, Artificial Sequence
  Synthetic peptide
DVQLQESGGDLVQPGGSLRLSCAASGFTLDYYAIGWFRQAPGKEREGVSCISSSDGSTYYADSVKGRFTSSRDNAKNTVYLQMNSLKPEDTAVYYCAAVPSTYYNGSYYYTCHPGGMDYWGKGTQVTVSS

SEQ ID NO: 2 - protein, 130 aa, Artificial Sequence
  Synthetic peptide
DVQLQESGGGLVQPGGSLRLSCAVSGFTLDYYAIGWFRQAPGKEREGVSCISSSDGSTYYADSVKGRFTSSRDNAKNTVYLQMNSLKPEDTAVYYCAAVPSTYYSGTYYYNCHPGGMDYWGKGTQVTVSS

SEQ ID NO: 3 - protein, 130 aa, Artificial Sequence
  Synthetic peptide
DVQLQESGGGLVQPGGSLRLSCAASGLTLDYYTIGWFRQAPGKEREGVSCISSSDDSTYYADSVKGRFTISRDNAKNTVYLQMNSLKPEDTAVYYCATAPGTYYKGSYYPMCHYYGMDYWGKGTQVTVSS

SEQ ID NO: 4 - protein, 130 aa, Artificial Sequence
  Synthetic peptide
DVQLQESGGGLVQPGGSLRLSCAVSGFTLDYYAIGWFRQAPGKEREGVACISSSDGTTYYADSVKGRFTISRDNAKNTVYLQMNSLKPEDTAVYYCATRPLTYYSGSYYTTCSDYGMDYWGKGTLVTVSS

SEQ ID NO: 5 - protein, 130 aa, Artificial Sequence
  Synthetic peptide
DVQLQESGGGLVQPGGSLRLSCAASGFTLDYYAIGWFRQAPGKEREGVSCISNSDGSTYYADSVKGRFTTSRDNAKNTVYLQMNSLKPEDTAVYYCAAVPSTYYSGSYYYTCHPGGMDYWGKGTQVTVSS

SEQ ID NO: 6 - protein, 130 aa, Artificial Sequence
  Synthetic peptide
DVQLQESGGGLVQSGGSLRLSCAASGFTLDYYAIGWFRQAPGKEREGVSCISNSDGSTYYADSVKGRFTTSRDNAKNTVYLQMNSLKPEDTAVYYCAAVPSTYYSGSYYYTCHPGGMDYWGKGTQVTVSS

SEQ ID NO: 7 - protein, 130 aa, Artificial Sequence
  Synthetic peptide
DVQLQESGGGLVQPGGSLRLSCAASGFTLDYYAIGWFRQAPGKEREGVSCISNSDGSTYYADSVKGRFTTSRDNAKNTVYLQMNSLKPEDTAVYYCAAVPSTYYSGSYYYTCHPGGMDYWGKGTLVTVSS

SEQ ID NO: 8 - protein, 130 aa, Artificial Sequence
  Synthetic peptide
DVQLQESGGGLVQPGGSLRLSCAASGFTLDYYAIGWFRQAPGKEREGVSCISNSGGSTYYADSVKGRFTTSRDNAKNTVYLQMNSLKPEDTAVYYCAAVPSTYYSGSYYYTCHPGGMDYWGKGTQVTVSS

SEQ ID NO: 9 - protein, 130 aa, Artificial Sequence
  Synthetic peptide
DVQLQESGGGLVQSGGSLRLSCAASGFTLDYYAIGWFRQAPGKEREGVSCITNSDGSTYYADSVKGRFTTSRDNAKNTVYLQMNSLKPEDTAVYYCASFPSTYYSGSYYYTCHPGGMDYWGKGTQVTVSS

SEQ ID NO: 10 - protein, 133 aa, Artificial Sequence
  Synthetic peptide
DVQLQESGGGLVQPGGSLRLSCAASGFTLDYYAIGWFRQAPGKEREGVSCISSSDGSTYYADSVKGRFTISRDNAKNTVYLQMNSLKPDDTAVYYCAAALSEGGYTIDGSSWCYHSVYGMDYWGKGTQVTVSS

SEQ ID NO: 11 - protein, 130 aa, Artificial Sequence
  Synthetic peptide
DVQLQESGGGSVEAGGSLRLSCAASGVTLDYYAIGWFRQAPGKEREGVSCISSSDGSTYYADSVKGRFTTSRDNAKNTVYLQMNSLKPEDTADYYCAAVPSTYYSGTYYYNCHPGAMHYWGKGTQVTVSS

SEQ ID NO: 12 - protein, 130 aa, Artificial Sequence [sequence omitted]

SEQ ID NO: 13 - protein, 130 aa, Artificial Sequence
  Synthetic peptide
DVQLQESGGGLVQPGGSLRLSCAASGLTLDYYAIGWFRQAPGKEREGVSCISSSDGSTYYADSVKGRFTTSRDNAKNTVYLQMNSLKPEDTAVYYCAAVPSTYYSGTYYYTCHPGGMDYWGKGTLVTVSS

SEQ ID NO: 14 - protein, 8 aa, Artificial Sequence
  Synthetic peptide
TLDYYAIG

SEQ ID NO: 15 - protein, 8 aa, Artificial Sequence
  Synthetic peptide
TLDYYTIG

SEQ ID NO: 16 - protein, 10 aa, Artificial Sequence
  Synthetic peptide
CISSSDGSTY

SEQ ID NO: 17 - protein, 10 aa, Artificial Sequence
  Synthetic peptide
CISSSDDSTY

SEQ ID NO: 18 - protein, 10 aa, Artificial Sequence
  Synthetic peptide
CISSSDGTTY

SEQ ID NO: 19 - protein, 10 aa, Artificial Sequence
  Synthetic peptide
CISNSDGSTY

SEQ ID NO: 20 - protein, 10 aa, Artificial Sequence
  Synthetic peptide
CISNSGGSTY

SEQ ID NO: 21 - protein, 10 aa, Artificial Sequence
  Synthetic peptide
CITNSDGSTY

SEQ ID NO: 22 - protein, 20 aa, Artificial Sequence
  Synthetic peptide
VPSTYYNGSYYYTCHPGGMD

SEQ ID NO: 23 - protein, 20 aa, Artificial Sequence
  Synthetic peptide
VPSTYYSGTYYYNCHPGGMD

SEQ ID NO: 24 - protein, 20 aa, Artificial Sequence
  Synthetic peptide
APGTYYKGSYYPMCHYYGMD

SEQ ID NO: 25 - protein, 20 aa, Artificial Sequence
  Synthetic peptide
RPLTYYSGSYYTTCSDYGMD

SEQ ID NO: 26 - protein, 20 aa, Artificial Sequence
  Synthetic peptide
VPSTYYSGSYYYTCHPGGMD

SEQ ID NO: 27 - protein, 20 aa, Artificial Sequence
  Synthetic peptide
FPSTYYSGSYYYTCHPGGMD

SEQ ID NO: 28 - protein, 23 aa, Artificial Sequence
  Synthetic peptide
ALSEGGYTIDGSSWCYHSVYGMD

SEQ ID NO: 29 - protein, 20 aa, Artificial Sequence
  Synthetic peptide
VPSTYYSGTYYYNCHPGAMH

SEQ ID NO: 30 - protein, 20 aa, Artificial Sequence
  Synthetic peptide
VPSTYYSGTYYYTCHPGGMD

SEQ ID NO: 31 - protein, 119 aa, Artificial Sequence
  Synthetic peptide
MAQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTSS

SEQ ID NO: 32 - protein, 125 aa, Artificial Sequence
  Synthetic peptide
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSS

SEQ ID NO: 33 - protein, 223 aa, Artificial Sequence
  Synthetic peptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 34 - DNA, 23 bp, Artificial Sequence
  Synthetic oligonucleotide
gtcctggctgctcttctacaagg

SEQ ID NO: 35 - DNA, 23 bp, Artificial Sequence
  Synthetic oligonucleotide
ggtacgtgctgttgaactgttcc

SEQ ID NO: 36 - DNA, 41 bp, Artificial Sequence
  Synthetic oligonucleotide
ccggccatggctgatgtgcagctgcaggagtctggrggagg

SEQ ID NO: 37 - DNA, 31 bp, Artificial Sequence
  Synthetic oligonucleotide
gtgcggccgctgaggagacggtgacctgggt

SEQ ID NO: 38 - DNA, 24 bp, Artificial Sequence
  Synthetic oligonucleotide
cgtatgttgtgtggaattgtgagc

SEQ ID NO: 39 - DNA, 39 bp, Artificial Sequence
  Synthetic oligonucleotide
tatctctcgagaaaagagatgtgcagctgcaggagtctg

SEQ ID NO: 40 - DNA, 40 bp, Artificial Sequence
  Synthetic oligonucleotide
ttgttctagattagtgatggtgatgatgatgtgcggccgc

(19) United States
(12) Patent Application Publication
(10) Pub. No.: US 2023/0235027 A1
(43) Pub. Date: Jul. 27, 2023

(54) NANOBODIES DIRECTED TO CORONAVIRUS SPIKE PROTEIN RECEPTOR BINDING DOMAIN AND USES THEREOF

(71) Applicant: The USA, as represented by the Secretary, Dept. of Health and Human Services, Bethesda, MD (US)

(72) Inventors: David Brody, Bethesda, MD (US); Thomas J. Esparza, Frederick, MD (US)

(21) Appl. No.: 18/011,767

(22) PCT Filed: Jul. 23, 2021
(86) PCT No.: PCT/US2021/042883

§ 371 Date: Dec. 20, 2022

Related U.S. Application Data
(60) Provisional application No. 63/055,865, filed on Jul. 23, 2020.

(51) Int. Cl. C07K 16/10; A01P 1/00; A61P 11/06; A01N 63/50; A61P 31/14; A61K 39/00

(52) U.S. Cl. CPC C07K 16/10; A01P 1/00; A61P 11/06; A01N 63/50; A61P 31/14; C07K 2317/34; C07K 2317/92; A61K 2039/54; C07K 2317/569; C07K 2317/76

(57) ABSTRACT

The invention generally relates to VHH domains that specifically bind a severe acute respiratory syndrome coronavirus spike protein receptor binding domain, corresponding expression vectors and host cells, and methods of treatment using such VHH domains.

### CROSS-REFERENCE TO RELATED APPLICATIONS

This application claims priority to U.S. Provisional Application No. 63/055,865, filed Jul. 23, 2020, which is incorporated herein by reference in its entirety.

### STATEMENT REGARDING FEDERALLY SPONSORED RESEARCH

This project was supported by the National Institutes of Health Intramural Research Program within the National Institute of Neurological Disorders and Stroke, the Uniformed Services University of the Health Sciences, and the Henry M. Jackson Foundation for the Advancement of Military Medicine.

### SEQUENCE LISTING STATEMENT

A computer readable form of the Sequence Listing is filed with this application by electronic submission and is incorporated into this application by reference in its entirety. The Sequence Listing is contained in the file created on Jul. 22, 2021, having the file name “20-1098-WO_Sequence-Listing_ST25.txt” and is 26 kilobytes in size.

### BACKGROUND OF THE INVENTION

### Field of the Invention

This disclosure generally relates to VHH domains (or nanobodies) that specifically bind a severe acute respiratory syndrome coronavirus spike protein receptor binding domain, corresponding expression vectors and host cells, and methods of treatment using such VHH domains.

### Description of Related Art

Coronaviruses are positive sense, single-stranded RNA viruses. There are seven types of coronaviruses known to infect humans, including the recent 2019 severe acute respiratory syndrome coronavirus 2 (SARS-CoV-2) (Cui, et al., Nat Rev Microbiol. 2019; 17(3):181-92. Epub 2018/12/12. doi: PubMed PMID: 30531947; PubMed Central PMCID: PMCPMC7097006; Zhou, et al., Nature. 2020; 579(7798):270-3. Epub 2020/02/06. doi: PubMed PMID: 32015507; PubMed Central PMCID: PMCPMC7095418). Patients infected with these viruses develop respiratory symptoms of various severity and outcomes. Since the beginning of the century, there have been three major world-wide health crises caused by coronaviruses: the 2003 SARS-CoV-1 outbreak, the 2012 MERS-CoV outbreak, and the 2019 SARS-CoV-2 outbreak (Huang, et al., Lancet. 2020; 395(10223):497-506. Epub 2020/01/28. doi: PubMed PMID: 31986264). To date, hundreds of thousands of people have succumbed to the virus during these outbreaks.

The SARS-CoV-2 virus gains entry to human cells via the angiotensin converting enzyme 2 (ACE2) receptor by the SARS-CoV-2 receptor binding domain (RBD) of the spike protein on the viral surface (Yuan, et al., Science. 2020. Epub 2020/04/05. doi: PubMed PMID: 32245784; Li, et al., Nature. 2003; 426(6965):450-4. Epub 2003/12/04. doi: PubMed PMID: 14647384; PubMed Central PMCID: PMCPMC7095016; Li, et al., EMBO J. 2005; 24(8):1634-43. Epub 2005/03/26. doi: PubMed PMID: 15791205; PubMed Central PMCID: PMCPMC1142572; Shang, et al., Nature. 2020; 581(7807):221-4. Epub 2020/04/01. doi: PubMed PMID: 32225175; PubMed Central PMCID: PMCPMC7328981; Yan, et al., Science. 2020; 367(6485):1444-8. Epub 2020/03/07. PubMed PMID: 32132184; PubMed Central PMCID: PMCPMC7164635) (see e.g., FIG. 1).

Many of the antibodies considered for diagnostic, research, and therapeutic applications have been conventional immunoglobulins (IgG). The use of IgGs as therapeutics, while successful in many diseases, is known to have potential pitfalls due to the risk of receptor-mediated immunological reactions (Descotes, MAbs. 2009; 1(2):104-11. Epub 2010/01/12. doi: PubMed PMID: 20061816; PubMed Central PMCID: PMCPMC2725414). There remains a substantial need for safe, preventative, and acute treatments for SARS-CoV-2.

### SUMMARY OF THE INVENTION

It is against the above background that the present invention provides certain advantages over the prior art.

Although this invention as disclosed herein is not limited to specific advantages or functionalities (such for example, VHH domains that specifically bind a severe acute respiratory syndrome coronavirus spike protein receptor binding domain, corresponding expression vectors and host cells, and methods of treatment using such VHH domains), the invention provides a VHH domain that specifically binds a severe acute respiratory syndrome coronavirus spike protein receptor binding domain, wherein the amino acid sequence of the VHH domain comprises any one of SEQ ID NOs:1-13.

Also disclosed herein is a VHH domain that specifically binds a severe acute respiratory syndrome coronavirus spike protein receptor binding domain, wherein the VHH domain comprises:
(a) a CDR1 as set forth in SEQ ID NO:14, a CDR2 as set forth in SEQ ID NO:16, and CDR3 as set forth in SEQ ID NO:22;
(b) a CDR1 as set forth in SEQ ID NO:14, a CDR2 as set forth in SEQ ID NO:16, and a CDR3 as set forth in SEQ ID NO:23;
(c) a CDR1 as set forth in SEQ ID NO:15, a CDR2 as set forth in SEQ ID NO:17, and a CDR3 as set forth in SEQ ID NO:24;
(d) a CDR1 as set forth in SEQ ID NO:14, a CDR2 as set forth in SEQ ID NO:18, and a CDR3 as set forth in SEQ ID NO:25;
(e) a CDR1 as set forth in SEQ ID NO:14, a CDR2 as set forth in SEQ ID NO:19, and a CDR3 as set forth in SEQ ID NO:26;
(f) a CDR1 as set forth in SEQ ID NO:14, a CDR2 as set forth in SEQ ID NO:20, and a CDR3 as set forth in SEQ ID NO:26;
(g) a CDR1 as set forth in SEQ ID NO:14, a CDR2 as set forth in SEQ ID NO:21, and a CDR3 as set forth in SEQ ID NO:27;
(h) a CDR1 as set forth in SEQ ID NO:14, a CDR2 as set forth in SEQ ID NO:16, and a CDR3 as set forth in SEQ ID NO:28;
(i) a CDR1 as set forth in SEQ ID NO:14, a CDR2 as set forth in SEQ ID NO:16, and a CDR3 as set forth in SEQ ID NO:29; or
(j) a CDR1 as set forth in SEQ ID NO:14, a CDR2 as set forth in SEQ ID NO:16, and a CDR3 as set forth in SEQ ID NO:30.

In one aspect of the VHH domain, the severe acute respiratory syndrome coronavirus spike protein binding domain is a severe acute respiratory syndrome coronavirus 2 (SARS-CoV-2) spike protein binding domain.

In one aspect, the VHH domain has a binding affinity to SARS-CoV-2) spike protein binding domain of at least 3 nM.

In one aspect, the affinity is assessed using surface plasmon resonance (SPR) or flow cytometry.

The invention also provides a nucleic acid, comprising a nucleotide sequence encoding the amino acid sequence of the VHH domain, a vector comprising the nucleic acid, and a host cell comprising the expression vector.

The invention also provides a vector, comprising the nucleic acid disclosed herein.

The invention also provides a host cell, comprising the expression vector disclosed herein.

The invention also provides an immunoconjugate, comprising (i) the VHH domain disclosed herein and (b) a conjugating part selected from a detectable moiety, a drug, a toxin, or a cytokine.

In one aspect, the detectable moiety is selected from fluorophores, immuno-histochemical tracers, positron emission tomography (PET) tracers, near infrared spectrometer (NIR) probes, single-photon emission computerized tomography (SPECT), magnetic particle imaging, magnetic resonance imaging contrast agents, ultrasound contrast agents, and radio-isotopes.

The invention also provides a pharmaceutical composition, comprising
(a) a therapeutically effective amount of the VHH domain or the immunoconjugate disclosed herein; and
(b) a pharmaceutically acceptable carrier.

The invention also provides a VHH domain that specifically binds a severe acute respiratory syndrome coronavirus spike protein receptor binding domain, wherein the VHH domain comprises a CDR1 as set forth in SEQ ID NOs:14 or 15; CDR 2 as set forth in any one of SEQ ID NOs:16 17, 18, 19, 20, or 21; and CDR3 as set forth in any one of SEQ ID NOs:23-30.

The invention also provides VHH domains that bind to the severe acute respiratory syndrome coronavirus spike protein receptor binding domain comprising SEQ ID NO:33 (amino acids Arg 319-Phe 541 of the amino acid sequence shown in Accession #QHD43416.1).

The VHH domains and immunoconjugates disclosed herein can be included in a pharmaceutical composition, comprising a therapeutically effective amount of the VHH domain or the immunoconjugate and a pharmaceutically acceptable carrier.

The invention also provides a method of treating severe acute respiratory syndrome coronavirus, the method comprising administering to a subject a therapeutically effective amount of the pharmaceutical composition comprising a therapeutically effective amount of the VHH domain or the immunoconjugate disclosed herein and a pharmaceutically acceptable carrier.

The invention also provides a method of preventing severe acute respiratory syndrome coronavirus, the method comprising administering to a subject a therapeutically effective amount of the pharmaceutical composition comprising a therapeutically effective amount of the VHH domain or the immunoconjugate disclosed herein and a pharmaceutically acceptable carrier.

The invention also provides a method of ameliorating severe acute respiratory syndrome coronavirus, the method comprising administering to a subject a therapeutically effective amount of the pharmaceutical composition comprising a therapeutically effective amount of the VHH domain or the immunoconjugate disclosed herein and a pharmaceutically acceptable carrier.

In one aspect of the methods disclosed herein, the severe acute respiratory syndrome coronavirus is severe acute respiratory syndrome coronavirus 2 (SARS-CoV-2).

In one aspect of the methods disclosed herein, the VHH domain is administered parenterally.

In one aspect of the methods disclosed herein, the parenteral administration is intravenous, intradermal, intrathecal, inhalation, transdermal (topical), intraocular, intramuscular, subcutaneous, pulmonary delivery, and/or transmucosal administration.

The invention also provides a method of diagnosis of severe acute respiratory syndrome coronavirus in a subject using the VHH domain disclosed herein.

In one aspect of the methods disclosed herein, the diagnosis is in vitro and/or in vivo.

The invention also provides a method for diagnosing a severe acute respiratory syndrome coronavirus infection in a patient, comprising detecting in a sample from the patient a spike protein receptor binding domain from a severe acute respiratory syndrome coronavirus, wherein the sample is contacted with the VHH domain disclosed herein.

The invention also provides a method for detecting a severe acute respiratory syndrome coronavirus on a contaminated surface, comprising detecting in a sample from the contaminated surface a spike protein receptor binding domain from a severe acute respiratory syndrome coronavirus wherein the sample is contacted with the VHH domain disclosed herein.

The invention also provides a method of decontaminating a severe acute respiratory syndrome coronavirus contaminated surface, comprising: contacting the surface which is contaminated with a composition comprising the VHH domain disclosed herein for sufficient time to substantially reduce the virus on the surface.

In one aspect of the methods disclosed herein, the severe acute respiratory syndrome coronavirus is severe acute respiratory syndrome coronavirus 2 (SARS-CoV-2).

The invention also provides a binding polypeptide complex, comprising a dimer of a first VHH, comprising the VHH domain disclosed herein and a second VHH, comprising the VHH domain disclosed herein, wherein the first VHH and the second VHH domains are linked by a dimerization domain.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic illustrating the structure of SARS-CoV-2 spike protein, with receptor binding domain in contact with the human ACE2 protein on the surface of a lung epithelial cell.

FIGS. 2A-2D show isolation of nanobodies binding SARS-CoV-2 spike protein. FIG. 2A illustrates the step where an adult llama was immunized 5 times over 28 days with purified, recombinant SARS-Cov-2 spike protein. On day 35 after the first immunization (7 days after last immunization), llama blood was obtained through a central line, B-cells were isolated, the single heavy-chain variable domains (nanobodies) of the llama antibodies were amplified and cloned to construct a recombinant DNA library containing more than 10⁸ clones. The library of clones was expressed in a phage display format, in which each phage expresses one nanobody on its surface and also contains the DNA sequence encoding that nanobody. Immunopanning was performed to isolate candidate nanobodies for expression and validation studies. FIG. 2B shows the reagents required for characterization and validation including recombinant human ACE2, recombinant SARS-Cov-2 receptor binding domain (RBD), and recombinant SARS-Cov-2 Spike protein (S1). Single bands on the protein-stained SDS-PAGE gel indicated purity and appropriate size. FIGS. 2C-2D illustrate validation that recombinant SARS-Cov-2 RBD and SARS-Cov-2 Spike S1 bound with high affinity to recombinant human ACE2. This high affinity, saturable binding indicated that all 3 recombinant proteins were appropriately folded in vitro.

FIGS. 3A-3C show the selection strategy for isolation of nanobody candidates which bind to the RBD:ACE2 interaction surface. FIG. 3A shows the step where ACE2 was immobilized in a standard radioimmunoassay tube and the surface blocked with non-specific protein. Biotinylated-RBD, was incubated with the nanobody phage library and then added to the immuno-tube and allowed to interact with the immobilized ACE2. Biotinylated-RBD with no associated nanobodies, or with nanobody associations which did not block the ACE2 binding domain, bound the immobilized ACE2. FIG. 3B shows the step where biotinylated-RBD with associated nanobodies that blocked the ACE2 binding domain remained in solution and were recovered using streptavidin-coated magnetic particles that bind to the biotin. FIG. 3C shows the step where nanobodies which did not bind to RBD were removed during washing of the magnetic beads. This method allowed for specific enrichment of nanobodies which both bind to the RBD and compete for the RBD-ACE2 binding surface.

FIG. 4 shows the protein sequences for novel nanobodies that bind to the SARS-Cov-2 spike protein receptor binding domain. Highlighting indicates sequence diversity with NIH-CoVnb-112 set as the reference sequence for comparison. For reference, two previously published nanobody sequences (VHH72 from Wrapp, et al. Cell. 2020; 181(5):1004-15 e15. Epub 2020/05/07. doi: PubMed PMID: 32375025; PubMed Central PMCID: PMCPMC7199733; and Ty1 from Hanke, et al. bioRxiv. 2020. Epub 06/02/2020. doi) have clearly distinct sequences. Both VHH72 and Ty1 have shorter CDR3 domains (represented in NIH-CoVnb-112 by amino acids 99-120).

FIGS. 5A-5F show affinity binding curves of isolated anti-SARS-CoV-2 RBD nanobodies. Using Biolayer Interferometry on a BioForte Octet Red96 system, association and dissociation rates were determined by immobilizing biotinylated-RBD onto streptavidin coated optical sensors (FIGS. 5A-5E). The RBD-bound sensors were incubated in specific concentrations of purified candidate nanobodies for a period of time to allow association. The sensors are then moved to nanobody-free solution and allowed to dissociate over a period of time. Curve fitting using a 1:1 interaction model allows for the affinity constant (K_{D}) to be measured for each nanobody as detailed in (FIG. 5F). FIG. 5G shows the surface plasmon resonance affinity measurement of NIH-CoVnb-112 binding RBD. FIGS. 5H-5I show characterization of NIH-CoVnb-112 by circular dichroism. FIG. 5H shows a representative CD curve for NIH-CoVnb-112. FIG. 5I shows that reversible folding was monitored using circular dichroism using a Jasco J-815 spectropolarimeter at 205 nm during a heating-cooling cycle over 25° C. to 85° C. at a rate of 2.5° C./min. The inflection point at 74.4° C. indicates the melting temperature. Using the delta between the heating and cooling curves is used to calculate a 73% refolding rate for NIH-CoVnb-112 which is indicative of a highly stable structure.

FIGS. 6A-6B show competitive inhibition of ACE2 and RBD binding using anti-SARS-CoV-2 RBD nanobodies. FIG. 6A shows RBD coated ELISA plates were blocked with non-specific protein and incubated with dilutions of each candidate anti-SARS-CoV-2 RBD nanobody. Biotinylated-ACE2 was added to each well and allowed to bind to unoccupied RBD. The ELISA was then developed using a standard streptavidin-HRP and tetramethylbenzidine reaction. Unoccupied RBD allows for a positive reaction signal which is suppressed in the presence of bound competitive nanobody. NIH-CoVnb-112 produced the greatest inhibition of ACE2 binding with an EC₅₀ of 0.02 micrograms/mL (1.11 nM). FIG. 6B shows comparable findings using the commercially available Genscript SARS-CoV-2 neutralization surrogate assay.

FIGS. 7A-7B show interaction of NIH-CoVnb-112 with SARS-Cov-2 Spike Protein RBD variants. FIG. 7A shows RBD “wild type” and 3 variant forms of the RBD coated ELISA plates that were blocked with non-specific protein and incubated with dilutions of each the lead candidate anti-SARS-CoV-2 RBD nanobody NIH-CoVnb-112. Biotinylated-ACE2 was added to each well and allowed to bind to unoccupied RBD. The ELISA was then developed using a conventional streptavidin-HRP and tetramethylbenzidine reaction. Unoccupied RBD allows for a positive reaction signal which is suppressed in the presence of bound competitive nanobody. NIH-CoVnb-112 produced the inhibition of ACE2 binding to each of the variants with a similar EC₅₀ of 0.02 micrograms/mL (1.11 nM). FIG. 7B shows binding of NIH-CoVnb-112 to RBD “wild type” and 3 variant forms of the RBD had similar affinity, with half maximal binding at approximately 0.01 micrograms/mL. FIGS. 7C-7E show that NIH-CoVnb-112 binds to SARS-CoV-2 RBD at a distinct epitope from that bound by VHH72 and does not bind to SARS-CoV-1 RBD.

FIG. 8 shows HEK293T+ACE-2 cells transduced with SARS-CoV-2 spike (ER retention signal removed) pseudotyped lentivirus CD512-EF1a-RFP). Lentivirus and nanobody (NB) at 0.3, 1, 3, or 10 micrograms per ml were incubated at 37 C for 30 minutes, and then added to media of HEK293 cells transfected with human ACE2. The pseudotyped lentivirus contains a red fluorescent protein gene, which is expressed in infected cells (top panels). The virus was added at a calculated multiplicity of infection (MOI)=0.5, such that approximately ½ of the cells should be infected. By flow cytometry (bottom panel), 42% of the cells were infected in the absence of nanobody at 2 days after infection, but there was substantially less infection when incubated with increasing concentrations of NIH-CoVnb-112 nanobody (bottom panels).

FIGS. 9A-9E show NIH-CoVnb-112 stability and potent inhibition of SARS-CoV-2 pseudovirus following nebulization. FIG. 9A is a schematic demonstrating that an Aerogen Solo High-Performance Vibrating Mesh nebulizer was placed in line with a custom glass bead condenser to allow for collection of the nanobody following nebulization. (Image element courtesy of Aerogen.) FIG. 9B shows a pre-nebulization and post-nebulization sample of purified NIH-CoVnb-112 analyzed by SDS-PAGE gel. The dominant band for each sample remained at approximately 14 kDa indicating no detectable degradation or aggregation of NIH-CoVnb-112 following nebulization. FIG. 9C shows that size exclusion chromatography demonstrated a prominent peak eluting at 13.5 mL elution volume in both pre and post-nebulization samples. FIG. 9D shows a fluorescence reporter assay utilizing SARS-CoV-2 spike protein pseudotyped lentivirus used to demonstrate potent inhibition of the RBD:ACE2 interaction. HEK293 cells overexpressing human ACE2 were cultured for 24 h with pseudotyped virus which was pretreated with NIH-CoVnb-112 at different concentrations. Inhibition of the spike RBD occurred when the virus was not able to transduce the HEK293-ACE2 cells and subsequently produce RFP reporter protein. FIG. 9E shows HEK293-ACE2 cells following 48 hr incubation analyzed by flow cytometry to quantify the fluorescence level. NIH-CoVnb-112 potently inhibited viral transduction both pre and post-nebulization with an EC50 of 0.323 μg/mL (23.1 nM) and 0.116 μg/mL (8.3 nM) respectively.

FIGS. 10A-10B show that NIH-CoVnb-112 is highly stable during controlled incubation with human plasma and albumin. To determine if NIH-CoVnb-112 is sensitive to plasma conditions a controlled incubation was performed at 37° C. with mixing. Measurement of binding, following incubation for 24 or 48 hrs, of NIH-CoVnb-112 in (FIG. 10A) pooled human plasma or (FIG. 10B) recombinant human albumin alone to mimic conditions which could lead to degradation and loss of function. NIH-CoVnb-112 was additionally spiked into pooled human plasma or recombinant human albumin immediately prior to measurement by biolayer interferometry.

FIG. 11A shows pseudovirus neutralization for a number of variants of NIH-CoVnb-112 following nebulization-recovery to demonstrate effective potency retention. FIG. 11B shows the measured affinity constant values for NIH-CoVnb-112 against various RBD mutant proteins demonstrating broad binding potential notwithstanding the range of tested mutations.

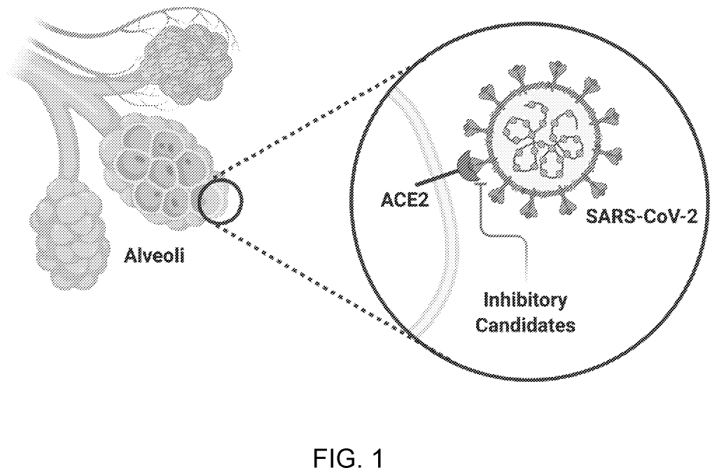

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE DISCLOSURE

All publications, patents and patent applications cited herein are hereby expressly incorporated by reference for all purposes.

Before describing the present invention in detail, a number of terms will be defined. Unless otherwise required by context, singular terms shall include pluralities and plural terms shall include the singular. For example, the singular forms “a,” “an,” and “the” include plural referents unless the context clearly dictates otherwise.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

The camelid family, which includes llamas, produce a subclass of IgGs which possess a single heavy-chain variable domain. This heavy-chain variable domain has demonstrated the ability to function as an independent antigen-binding domain with similar affinity as a conventional IgG. These heavy chain variable domains can be expressed as a single domain, known as a VHH or nanobody, with a molecular weight 10% of the full IgG.

The terms “VHH domain” and “nanobody,” are used herein interchangeable. The terms are used in their broadest sense, and not limited to a specific biological source or to a specific method of preparation.

Nanobodies generally display superior solubility, solution stability, temperature stability, strong penetration into tissues, are easily manipulated with recombinant molecular biology methods, and possess robust environmental resilience to conditions detrimental to conventional IgGs. In addition, nanobodies are weakly immunogenic which reduces the likelihood of adverse effects compared to other single domain antibody such as those derived from sharks or synthetic platforms.

In some embodiments, the amino acid sequences of the disclosure correspond to amino acid sequences of naturally occurring VHH domains, but that have been “humanized,” i.e., by replacing one or more amino acid residues in the amino acid sequence of the naturally occurring VHH sequence by one or more of the amino acid residues that occur at the corresponding positions in a VH domain from a conventional 4-chain antibody from a human being. This can be performed in a manner known in the art.

In some embodiments provided herein is a VHH domain that specifically binds a severe acute respiratory syndrome coronavirus spike protein receptor binding domain, wherein the amino acid sequence of the VHH domain comprises any one of SEQ ID NOs. 1-13. In some embodiments, the amino acid sequence of the VHH domain comprises SEQ ID NO: 12. In some embodiments, the disclosure provides VHH domain amino acid sequences having at least 80%, or at least 90%, or at least 95%, or at least 99% sequence identity to any one of the amino acid sequences set forth in SEQ ID NOs:1-13.

In particular embodiments, provided herein are VHH domain amino acid sequences with a conservative variant in which there are up to 10, up to 8, up to 5, and up to 3 amino acids substituted by amino acids having analogical or similar properties, compared to the amino acid sequence of the VHH domains disclosed herein.

In some embodiments provided herein is a VHH domain that specifically binds a severe acute respiratory syndrome coronavirus spike protein receptor binding domain, wherein the VHH domain comprises three complementarity determining regions (CDR1, CDR2, and CDR3). In some embodiments the VHH domain comprises,
(a) a CDR1 as set forth in SEQ ID NO:14, a CDR2 as set forth in SEQ ID NO:16, and CDR3 as set forth in SEQ ID NO:22;
(b) a CDR1 as set forth in SEQ ID NO:14, a CDR2 as set forth in SEQ ID NO:16, and a CDR3 as set forth in SEQ ID NO:23;
(c) a CDR1 as set forth in SEQ ID NO:15, a CDR2 as set forth in SEQ ID NO:17, and a CDR3 as set forth in SEQ ID NO:24;
(d) a CDR1 as set forth in SEQ ID NO:14, a CDR2 as set forth in SEQ ID NO:18, and a CDR3 as set forth in SEQ ID NO:25;
(e) a CDR1 as set forth in SEQ ID NO:14, a CDR2 as set forth in SEQ ID NO:19, and a CDR3 as set forth in SEQ ID NO:26;
(f) a CDR1 as set forth in SEQ ID NO:14, a CDR2 as set forth in SEQ ID NO:20, and a CDR3 as set forth in SEQ ID NO:26;
(g) a CDR1 as set forth in SEQ ID NO:14, a CDR2 as set forth in SEQ ID NO:21, and a CDR3 as set forth in SEQ ID NO:27;
(h) a CDR1 as set forth in SEQ ID NO:14, a CDR2 as set forth in SEQ ID NO:16, and a CDR3 as set forth in SEQ ID NO:28;
(i) a CDR1 as set forth in SEQ ID NO:14, a CDR2 as set forth in SEQ ID NO:16, and a CDR3 as set forth in SEQ ID NO:29; or
(j) a CDR1 as set forth in SEQ ID NO:14, a CDR2 as set forth in SEQ ID NO:16, and a CDR3 as set forth in SEQ ID NO:30.

The term “specifically binds” refers to a VHH domain that specifically binds to a molecule or a fragment thereof (e.g., antigen). A VHH domain that specifically binds a molecule or a fragment thereof can bind to other molecules with lower affinity as determined by, e.g., immunoassays, BIAcore, or other assays known in the art.

In particular embodiments, the VHH domain specifically binds to the severe acute respiratory syndrome coronavirus spike protein receptor binding domain. In particular embodiments, the severe acute respiratory syndrome coronavirus spike protein receptor binding domain is a severe acute respiratory syndrome coronavirus 2 (SARS-CoV-2) spike protein receptor binding domain. The nanobodies disclosed herein bind to the SARS-CoV-2 spike protein receptor binding domain and block spike protein interaction with the angiotensin converting enzyme 2 (ACE2) receptor.

Coronaviruses are positive-strand RNA viruses and the virion consists of a nucleocapsid core surrounded by an envelope containing three membrane proteins, spike (S), membrane (M) and envelope (E), which are common to all members of the genus. The S protein, which forms morphologically characteristic projections on the virion surface, mediates binding to host receptors and membrane fusion. The S protein of coronavirus is responsible for inducing host immune responses and virus neutralization by antibodies. The M and E proteins are important for viral assembly while N is important for viral RNA packaging.

The term “K_{D},” as used herein, refers to the dissociation constant of the interaction between a VHH domain disclosed herein and a target antigen. The VHH domain binds to a severe acute respiratory syndrome coronavirus spike protein receptor binding domain with a dissociation constant (K_{D}) of 10⁻⁵ to 10⁻¹² moles/liter or less, or 10⁻⁷ to 10⁻¹² moles/liter or less, or 10⁻³ to 10⁻¹² moles/liter, and/or with a binding affinity of at least 10⁷ M⁻¹, or at least 10⁸ M⁻¹, or at least 10⁹ M⁻¹, or at least 10¹² M⁻¹. Any K_{D} value greater than 10⁻⁴ liters/mole is generally considered to indicate non-specific binding. In some embodiments, VHH domains of the disclosure will bind to a desired antigen with an affinity less than 500 mM, or less than 200 nM, or less than 10 nM, or less than 500 pM. In some embodiments, the VHH domain has a binding affinity of at least 3 nM.

The dissociation constant (K_{D}) can be determined, for example, by surface plasmon resonance (SPR). Generally, surface plasmon resonance analysis measures real-time binding interactions between a ligand (a target antigen on a biosensor matrix) and an analyte by surface plasmon resonance using, for example, the BIAcore system (Pharmacia Biosensor; Piscataway, N.J.). Surface plasmon analysis can also be performed by immobilizing the analyte and presenting the ligand. Specific binding of a VHH domain that specifically binds an antigen or antigenic determinant to that antigen or antigenic determinant can also be determined in any suitable manner known in the art, including, for example, Scatchard analysis and/or competitive binding assays, such as radioimmunoassays (RIA), enzyme immunoassays (EIA), and sandwich competition assays, and different variants thereof known in the art. In some embodiments, the affinity of VHH domains disclosed herein is assessed using surface plasmon resonance (SPR) or flow cytometry.

The VHH domains disclosed herein can be employed in any known assay method, such as competitive binding assays, direct and indirect sandwich assays, and immunoprecipitation assays for the detection and quantitation of one or more target antigens. The VHH domains will bind the one or more target antigens with an affinity that is appropriate for the assay method being employed.

In some embodiments provided is a method of in vitro diagnosis of severe acute respiratory syndrome coronavirus in a subject using the VHH domains disclosed herein. The VHH domains disclosed herein can used in vitro in immunoassays in which they can be utilized in liquid phase or bound to a solid phase carrier. In addition, the VHH domains in these immunoassays can be detectably labeled in various ways. Examples of types of immunoassays which can utilize the VHH domains disclosed herein are flow cytometry, e.g., FACS, MACS, immunohistochemistry, competitive and non-competitive immunoassays in either a direct or indirect format.

The VHH domains disclosed herein can be conjugated to a drug or therapeutic agent that modifies a given biological response. Suitable drugs include chemical therapeutic agents, a protein or polypeptide possessing a desired biological activity for example, a toxin, a cytokine, a prothrombotic or antithrombotic agent, an anti-angiogenic agent, or a growth factor. Additionally, the VHH domains can be conjugated to therapeutic moieties such as a radioactive materials or macrocyclic chelators. In some embodiments provided is an immunoconjugate, comprising the VHH domains disclosed herein and a conjugating part selected from a drug, toxin, or cytokine.

In some embodiments disclosed herein is a method for diagnosing a severe acute respiratory syndrome coronavirus infection in a patient, comprising detecting in a sample from the patient a spike protein receptor binding domain from a severe acute respiratory syndrome coronavirus, wherein the sample is contacted with the VHH domains disclosed herein. In some embodiments, the sample is blood, serum, plasma, urine, feces, respiratory secretions, cerebrospinal fluid, or saliva.

The VHH domains disclosed herein are also useful for in vivo imaging and diagnostic applications. A VHH domain labeled with a detectable moiety can be administered to a patient, for example into the bloodstream, and the presence and location of the labeled VHH domain in the host assayed. The VHH domain can be labeled with any moiety that is detectable in a patient, whether by nuclear magnetic resonance, radiology, or other detection means known in the art. In some embodiments provided is an immunoconjugate comprising the VHH domains disclosed herein and a conjugating part comprising a detectable moiety. For example, the detectable moiety can be a radioisotope, such as ³H, ¹⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase. In some embodiments, the detectable moiety is selected from fluorophores, immuno-histochemical tracers, positron emission tomography (PET) tracers, near infrared spectrometer (NIR) probes, single-photon emission computerized tomography (SPECT), magnetic particle imaging, magnetic resonance imaging contrast agents, ultrasound contrast agents, and radio-isotopes.

In particular embodiments provided here is a nucleic acid comprising a nucleotide sequence encoding the amino acid sequence of the VHH domain, a vector comprising the nucleic acid, and a host cell comprising the expression vector.

In particular embodiments provided herein is a binding polypeptide complex comprising a dimer of a first VHH domain comprising the VHH domains disclosed herein and a second VHH domain comprising the VHH domains disclosed herein, wherein the first VHH and the second VHH domains are linked by a dimerization domain. In particular embodiments, the dimerization domain comprises one or more of C_{H}2, C_{H}3 or C_{H}4 antibody constant region domains, a short serine/glycine polypeptide, and/or a J chain.

Where the dimerization domains of the heavy chains comprise immunoglobulin heavy chain constant regions, the constant regions (C_{H} exons) can give further physiological functionality to the polypeptide binding complex. In particular, the immunoglobulin heavy chain constant domains can provide for, inter alia, complement fixation, macrophage activation and binding to Fc receptors, depending on the class or subclass of the antibody constant domains.

The term “patient” is intended to include human and non-human animals, particularly mammals.

The terms “treatment” or “treat” as used herein refer to both therapeutic treatment and prophylactic or preventative measures. Those in need of treatment include subjects having a severe acute respiratory syndrome coronavirus infection as well as those prone to having a severe acute respiratory syndrome coronavirus infection or those in a severe acute respiratory syndrome coronavirus infection is to be prevented.

In some embodiments, the methods disclosed herein relate to treating a patient for a severe acute respiratory syndrome coronavirus or preventing a severe acute respiratory syndrome coronavirus in a patient by administering the VHH domains disclosed herein. In some embodiments, the severe acute respiratory syndrome coronavirus is severe acute respiratory syndrome coronavirus 2 (SARS-CoV-2).

The terms “administration” or “administering” as used herein refer to providing, contacting, and/or delivering a compound or compounds by any appropriate route to achieve the desired effect.

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one VHH domain or immunoconjugate of the disclosure.

The terms “pharmaceutically acceptable carrier” or “physiologically acceptable carrier” as used herein refer to one or more formulation materials suitable for accomplishing or enhancing the delivery of one or more VHH domains of the disclosure.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the VHH domains disclosed herein. The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment, compositions can be prepared for storage by mixing the selected composition having the desired degree of purity with optional formulation agents in the form of a lyophilized cake or an aqueous solution. Further, the VHH domains disclosed herein can be formulated as a lyophilizate using appropriate excipients such as sucrose.

### Treatment of SARS-CoV2 Infection

In one embodiment provided herein is a method for treatment of SARS-CoV2 infection using inhaled pharmaceutical composition, comprising (a) a therapeutically effective amount of the VHH domain or the immunoconjugate disclosed herein; and (b) a pharmaceutically acceptable carrier.

In one particular embodiment, the nanobody pharmaceutical composition (therapeutic) can be formulated for nebulization or other appropriate methods for reaching the nasal passages, airways and lungs. The inhaled treatment can be administered in hospitals, urgent care centers, medical practitioners' offices, field stations, by home health workers, and potentially by patients themselves in much the same way that asthma treatments are delivered at present. The treatments can be easy to administer and relatively inexpensive given that the devices required to administer inhaled therapeutics have been developed and are widely used, and the nanobody therapeutic itself would be inexpensive to produce. Inhaled nanobody therapeutic can be combined with other inhaled therapeutics such as inhaled interferons, with distinct mechanisms of action.

In another particular embodiment, the nanobody pharmaceutical composition (therapeutic) can be formulated for intramuscular, subcutaneous and intravenous administration, and administered in hospitals, urgent care centers, medical practitioners' offices, field stations, or by home health workers. The treatments can be relatively inexpensive given that the nanobody therapeutic can be inexpensive to produce, compared to other intramuscular, subcutaneous and intravenous antibody therapeutics.

In yet another particular embodiment, the nanobody pharmaceutical composition (therapeutic) can be used in challenge vaccine studies involving healthy human volunteers who are administered a candidate vaccine, then challenged with a live, virulent pathogen in a controlled environment. Challenge vaccine studies can be used as a potential approach to accelerate the development of effective vaccines. However, challenge vaccine studies face regulatory and ethical hurdles when there is substantial risk to the healthy volunteers. The availability of an effective nanobody therapeutic as a rescue medication would reduce the risk to healthy volunteers. By analogy, the availability of Tamiflu substantially reduces the risk to healthy human volunteers during challenge vaccine studies of candidate influenza vaccines.

### Prevention of SARS-CoV2 Infection

In one embodiment provided herein is a method for prevention of SARS-CoV2 infection.

In one particular embodiment, an inhaled, an intramuscular, a subcutaneous, or an intravenous SARS-CoV2 nanobody pharmaceutical composition (therapeutic) can be given to intrinsically high risk individuals at appropriate intervals could reduce the likelihood and severity of infection. Intrinsically high risk individuals include the elderly, the immunocompromised, and those with comorbidities. SARS-CoV2 nanobody could allow intrinsically high risk individuals to obtain routine health care for other conditions with reduced risk of contracting SARS-CoV2 in health care settings. The nanobody therapeutic could allow intrinsically high risk individuals to participate in work, school, social interaction, and other valuable activities with reduced risk of contracting SARS-CoV2. Intramuscular, subcutaneous or intravenous SARS-CoV2 nanobody could reduce the likelihood and severity of infection under circumstances in which there is concern for exposure via non-respiratory routes. Nanobody therapeutic can be combined with other inhaled therapeutics such as inhaled interferons, with distinct mechanisms of action.

In one particular embodiment, an inhaled, an intramuscular, a subcutaneous, or an intravenous SARS-CoV2 nanobody pharmaceutical composition (therapeutic) can be given to individuals with anticipated exposure at appropriate intervals could reduce the likelihood and severity of infection for individuals with anticipated exposure. Individuals with anticipated exposure include health care workers, crowd control personnel, military service members serving in close quarters such as on ships, participants in indoor gatherings, caregivers living with infected individuals, and others. Intramuscular, subcutaneous and intravenous SARS-CoV2 nanobody could reduce the likelihood and severity of infection under circumstances in which there is concern for exposure via non-respiratory routes.

### Prevention of SARS-CoV2 Spread

In one embodiment provided herein is a method for prevention SARS-CoV2 spread. Inhalation of a SARS-CoV2 nanobody therapeutic in an infected individual would coat the airways and reduce the infectivity of virus spread through coughing, speaking, or breathing. Inhalation of a SARS-CoV2 nanobody therapeutic at appropriate intervals could allow infected individuals to return to work, school, military service, or other activities faster and more safely than is possible at present. Intramuscular, subcutaneous and intravenous SARS-CoV2 nanobody could reduce the likelihood and severity of spread via non-respiratory routes such as through gastrointestinal or other secretions.

Diagnosis of SARS-CoV2 Infection from Body Fluids

In one embodiment provided herein is a method for diagnosing of SARS-CoV2 infection from body fluids.

In one particular embodiment, the SARS-CoV2 nanobodies can be used for plate-based or other format ELISAs. The low cost of producing nanobodies compared with conventional IgGs would reduce the price of the ELISAs. The low cost could be especially beneficial for diagnostic testing in the developing world.

In another particular embodiment, laminar flow point of care assays using the SARS-CoV2 nanobodies can be more specific and less expensive than technologies based on conventional IgG. The excellent stability of nanobodies at extremes of temperature is another advantage for point-of-case use.

### Imaging of SARS-CoV2 in the Body

In one embodiment provided herein is a method for imaging of SARS-CoV2 in the body.

In one particular embodiment, SARS-CoV2 nanobodies conjugated to a suitable radioisotope such as ¹¹C, ¹⁸F, or ⁶⁴Cu could be used as PET ligands. After inhalation, intramuscular, subcutaneous or intravenous injection, the ligands would bind to SARS-CoV in tissues and allow visualization of the distribution of viral protein in the body. Such molecular contrast PET studies could further improve assessment of the pathophysiology of viral infection, including but not limited to understanding the effects on lungs, peripheral olfactory system, gastrointestinal system, heart, kidneys, muscles, and peripheral nerves. With additional blood brain barrier crossing technology (to be disclosed separately) imaging of the brain could be performed as well. PET has advantages over other imaging methods including high sensitivity, which translates into the requirement for very low concentrations of ligand.

In another particular embodiment, SARS-CoV2 nanobodies conjugated to a suitable MRI contrast agent such as gadolinium chelates, manganese chelates, or iron oxide nanoparticles could be used as MRI contrast agents. After inhalation, intramuscular, subcutaneous or intravenous injection, the contrast agents would bind to SARS-CoV in tissues and allow visualization of the distribution of viral protein in the body. Such molecular contrast MRI studies could further improve assessment of the pathophysiology of the viral infection, including but not limited to understanding the effects on lungs, peripheral olfactory system, gastrointestinal system, heart, kidneys, muscles, and peripheral nerves. With additional blood brain barrier crossing technology (to be disclosed separately) imaging of the brain could be performed as well. MRI has advantages over PET including better spatial resolution, lower cost, more widespread availability, and no radiation exposure.

In yet another particular embodiment, SARS-CoV2 nanobodies conjugated to a suitable ultrasound contrast agent such as liposomal microbubbles could be used as ultrasound contrast agents. After inhalation, intramuscular, subcutaneous or intravenous injection, the contrast agents would bind to SARS-CoV in tissues and allow visualization of the distribution of viral protein in parts of the body that are accessible to ultrasound. Such molecular contrast ultrasound studies could further improve assessment of the pathophysiology of the viral infection, including but not limited to understanding the effects on lungs, gastrointestinal system, heart, kidneys, muscles, and peripheral nerves. Ultrasound has advantages over other imaging methods including speed, portability, low cost, widespread availability, excellent time resolution, no radiation exposure, and few contraindications.

### Detection of SARS-CoV2 in the Environment

In one embodiment provided herein is a method for antibody-based tests that can be used to assess for SARS-CoV2 spike protein in the environment. The low cost and temperature stability of nanobodies would be advantageous in this setting.

### Decontamination of Surfaces Exposed to SARS-CoV2

In one embodiment provided herein is a method for decontamination of surfaces exposed to SARS-CoV2.

In one particular embodiment, spraying or wiping surfaces exposed to SARS-CoV2 with a solution containing high concentrations of SARS-CoV2 nanobody can rapidly decontaminate them and reduce risk of transmission by contact. This could enhance health care delivery by making scanners, operating rooms, and other medical facilities more rapidly available between patients. At present, it often requires 2 hours or more to clean facilities between patients to ensure safety. With a specific neutralization solution, the surfaces would be effectively rendered safe immediately. Surfaces on elevators, doors, public transportation facilities, and other indoor settings could be frequently decontaminated to enhance public safety. The low cost and low toxicity of the nanobody production would make this feasible.

The pharmaceutical compositions disclosed herein can be selected for parenteral delivery including intravenous, intradermal, intrathecal, inhalation, transdermal (topical), intraocular, intramuscular, subcutaneous, pulmonary delivery, and/or transmucosal administration. Intravenous, intramuscular, or subcutaneous treatment formulations can be used for example in early stage disease, as well as prevention in high risk individuals. It appears that late-stage disease is mediated more by immunological responses and less by virological pathogenesis, making it important to initiate virologically-targeted therapy early. Importantly, nanobody treatments would be substantially less expensive to produce than conventional monoclonal antibodies. Thus, it would be reasonable to initiate treatment with an inexpensive and widely available therapeutic early, even before it is clear whether or not an infection will become severe.

In one embodiment, the compositions can be selected for inhalation. For example, the VHH domains disclosed herein, can be formulated as a dry powder for inhalation, with a propellant for aerosol delivery. In yet another embodiment, solutions comprising the VHH domains disclosed herein can be nebulized. Inhalation has major advantages over other routes of administration. For example, Larios Mora et al. showed that nebulized treatment of newborn lambs with ALX-0171 reduced clinical, virological, and pathological manifestations of respiratory syncytial virus (Larios, et al. MAbs. 2018; 10(5):778-95. Epub 2018/05/08. doi: PubMed PMID: 29733750; PubMed Central PMCID: PMCPMC6150622). ALX-0171 is a trimeric nanobody therapy produced in Pischia pastoris that binds to the respiratory syncytial virus fusion protein. Nebulization was performed using a commercially available human-use Aeroneb Solo system, interfaced with a nose mask. The Aeroneb Solo nebulizer produced ˜3.3 micron particles, appropriate for deep lung delivery. Once daily nebulization resulted in concentrations of ALX-0171 in lung epithelial lining fluid that were >10 times higher than the in vitro EC₅₀ for respiratory syncytial virus. All treated lambs had undetectable infectious virus in lung epithelial lining fluid, which supports that the proposed route of administration could reduce infectivity. After nebulization, blood concentrations were ˜1,000 fold lower than lung epithelial lining fluid concentrations, which is important because low blood concentrations likely reduce systemic toxicity and risk of host antibody formation. Of note, the parent monomeric nanobody to the respiratory syncytial virus fusion protein had an affinity of 17 nM, whereas the nanobody trimer ALX-0171 had an affinity of 0.113 nM. The trimeric ALX-0171 was produced by fermentation in Pischia pastoris with a yield of 7.5 grams per liter (Detalle, et al. Antimicrob Agents Chemother. 2016; 60(1):6-13. Epub 2015/10/07. doi: PubMed PMID: 26438495; PubMed Central PMCID: PMCPMC4704182).

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in the methods disclosed herein can be in the form of a pyrogen-free, parenterally acceptable, aqueous solution comprising the desired polypeptides in a pharmaceutically acceptable vehicle. A particularly suitable vehicle for parenteral injection is sterile distilled water in which the desired VHH domains disclosed herein are formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

It is also contemplated that certain formulations can be administered orally. For example, a capsule can be designed to release the active portion of the formulation at the point in the gastrointestinal tract when bioavailability is maximized and pre-systemic degradation is minimized. Additional agents can be included to facilitate absorption. Diluents, flavorings, low melting point waxes, vegetable oils, lubricants, suspending agents, tablet disintegrating agents, and binders can also be employed.

In particular embodiments provided herein is a method for detecting a severe acute respiratory syndrome coronavirus on a contaminated surface, comprising detecting in a sample from the contaminated surface a spike protein receptor binding protein from a severe acute respiratory syndrome coronavirus wherein the sample is contacted with the VHH domain disclosed herein.

In particular embodiments provided herein is a method of treating a severe acute respiratory syndrome coronavirus contaminated surface, comprising: contacting the surface which is contaminated with a composition comprising the VHH domain disclosed herein for sufficient time to substantially reduce the virus on the surface.

Without limiting the disclosure, a number of embodiments of the disclosure are described below for purpose of illustration.

TABLE 1
VHH sequences. Bold and Highlighted amino
acids illustrate CDRs.

| Identi- | |
|---|---|
| fier | Sequence |
| NIH- | DVQLQESGGDLVQPGGSLRLSCAASGF |
| CoVnb- | TLDYYAIGWFRQAPGKEREGVS |
| 101 | CISSSDGSTY |
| | YADSVKGRFTSSRDNAKNTVYLQMNSLKPEDTAVYYCAA |
| | VPSTYYNGSYYYTCHPGGMD |
| | YWGKGTQVTVSS |
| | (SEQ ID NO: 1) |
| NIH- | DVQLQESGGGLVQPGGSLRLSCAVSGF |
| CoVnb- | TLDYYAIGWFRQAPGKEREGVS |
| 102 | CISSSDGSTY |
| | YADSVKGRFTSSRDNAKNTVYLQMNSLKPEDTAVYYCAA |
| | VPSTYYSGTYYYNCHPGGMD |
| | YWGKGTQVTVSS |
| | (SEQ ID NO: 2) |
| NIH- | DVQLQESGGGLVQPGGSLRLSCAASGL |
| CoVnb- | TLDYYTIGWFRQAPGKEREGVS |
| 103 | CISSSDDSTY |
| | YADSVKGRFTISRDNAKNTVYLQMNSLKPEDTAVYYCAT |
| | APGTYYKGSYYPMCHYYGMD |
| | YWGKGTQVTVSS |
| | (SEQ ID NO: 3) |
| NIH- | DVQLQESGGGLVQPGGSLRLSCAVSGF |
| CoVnb- | TLDYYAIGWFRQAPGKEREGVA |
| 104 | CISSSDGTTY |
| | YADSVKGRFTISRDNAKNTVYLQMNSLKPEDTAVYYCAT |
| | RPLTYYSGSYYTTCSDYGMD |
| | YWGKGTLVTVSS |
| | (SEQ ID NO: 4) |
| NIH- | DVQLQESGGGLVQPGGSLRLSCAASGF |
| CoVnb- | TLDYYAIGWFRQAPGKEREGVS |
| 105 | CISNSDGSTY |
| | YADSVKGRFTTSRDNAKNTVYLQMNSLKPEDTAVYYCAA |
| | VPSTYYSGSYYYTCHPGGMD |
| | YWGKGTQVTVSS |
| | (SEQ ID NO: 5) |
| NIH- | DVQLQESGGGLVQSGGSLRLSCAASGF |
| CoVnb- | TLDYYAIGWFRQAPGKEREGVS |
| 106 | CISNSDGSTY |
| | YADSVKGRFTTSRDNAKNTVYLQMNSLKPEDTAVYYCAA |
| | VPSTYYSGSYYYTCHPGGMD |
| | YWGKGTQVTVSS |
| | (SEQ ID NO: 6) |
| NIH- | DVQLQESGGGLVQPGGSLRLSCAASGF |
| CoVnb- | TLDYYAIGWFRQAPGKEREGVS |
| 107 | CISNSDGSTY |
| | YADSVKGRFTTSRDNAKNTVYLQMNSLKPEDTAVYYCAA |
| | VPSTYYSGSYYYTCHPGGMD |
| | YWGKGTLVTVSS |
| | (SEQ ID NO: 7) |
| NIH- | DVQLQESGGGLVQPGGSLRLSCAASGF |
| CoVnb- | TLDYYAIGWFRQAPGKEREGVS |
| 108 | CISNSGGSTY |
| | YADSVKGRFTTSRDNAKNTVYLQMNSLKPEDTAVYYCAA |
| | VPSTYYSGSYYYTCHPGGMD |
| | YWGKGTQVTVSS |
| | SEQ ID NO: 8) |
| NIH- | DVQLQESGGGLVQSGGSLRLSCAASGF |
| CoVnb- | TLDYYAIGWFRQAPGKEREGVS |
| 109 | CITNSDGSTY |
| | YADSVKGRFTTSRDNAKNTVYLQMNSLKPEDTAVYYCAS |
| | FPSTYYSGSYYYTCHPGGMD |
| | YWGKGTQVTVSS |
| | (SEQ ID NO: 9) |
| NIH- | DVQLQESGGGLVQPGGSLRLSCAASGF |
| CoVnb- | TLDYYAIGWFRQAPGKEREGVS |
| 110 | CISSSDGSTY |
| | YADSVKGRFTISRDNAKNTVYLQMNSLKPDDTAVYYCAA |
| | ALSEGGYTIDGSSWCYHSVYGMD |
| | YWGKGTQVTVSS |
| | (SEQ ID NO: 10) |
| NIH- | DVQLQESGGGSVEAGGSLRLSCAASGV |
| CoVnb- | TLDYYAIGWFRQAPGKEREGVS |
| 111 | CISSSDGSTY |
| | YADSVKGRFTTSRDNAKNTVYLQMNSLKPEDTADYYCAA |
| | VPSTYYSGTYYYNCHPGAMH |
| | YWGKGTQVTVSS |
| | (SEQ ID NO: 11) |
| NIH- | DVQLQESGGGLVQPGGSLRLSCAASGL |
| CoVnb- | TLDYYAIGWFRQAPGKEREGVS |
| 112 | CISSSDGSTY |
| | YADSVKGRFTTSRDNAKNTVYLQMNSLKPEDTAVYYCAA |
| | VPSTYYSGTYYYTCHPGGMD |
| | YWGKGTQVTVSS |
| | (SEQ ID NO: 12) |
| NIH- | DVQLQESGGGLVQPGGSLRLSCAASGL |
| CoVnb- | TLDYYAIGWFRQAPGKEREGVS |
| 113 | CISSSDGSTY |
| | YADSVKGRFTTSRDNAKNTVYLQMNSLKPEDTAVYYCAA |
| | VPSTYYSGTYYYTCHPGGMD |
| | YWGKGTLVTVSS |
| | (SEQ ID NO: 13) |

TABLE 2
CDR amino acid sequences of the VHH
nanobodies

| Identi- | | | |
|---|---|---|---|
| fier | CDR1 | CDR2 | CDR3 |
| NIH- | TLDYYAIG | CISSSDGSTY | VPSTYYNGSYYYTCHP |
| CoVnb- | (SEQ ID | (SEQ ID | GGMD |
| 101 | NO: 14) | NO: 16) | (SEQ ID NO: 22) |
| NIH- | TLDYYAIG | CISSSDGSTY | VPSTYYSGTYYYNCHP |
| CoVnb- | (SEQ ID | (SEQ ID | GGMD |
| 102 | NO: 14) | NO: 16) | (SEQ ID NO: 23) |
| NIH- | TLDYYTIG | CISSSDDSTY | APGTYYKGSYYPMCHY |
| CoVnb- | (SEQ ID | (SEQ ID | YGMD |
| 103 | NO: 15) | NO: 17) | (SEQ ID NO: 24) |
| NIH- | TLDYYAIG | CISSSDGTTY | RPLTYYSGSYYTTCSD |
| CoVnb- | (SEQ ID | (SEQ ID | YGMD |
| 104 | NO: 14) | NO: 18) | (SEQ ID NO: 25) |
| NIH- | TLDYYAIG | CISNSDGSTY | VPSTYYSGSYYYTCHP |
| CoVnb- | (SEQ ID | (SEQ ID | GGMD |
| 105 | NO: 14) | NO: 19) | (SEQ ID NO: 26) |
| NIH- | TLDYYAIG | CISNSDGSTY | VPSTYYSGSYYYTCHP |
| CoVnb- | (SEQ ID | (SEQ ID | GGMD |
| 106 | NO: 14) | NO: 19) | (SEQ ID NO: 26) |
| NIH- | TLDYYAIG | CISNSDGSTY | VPSTYYSGSYYYTCHP |
| CoVnb- | (SEQ ID | (SEQ ID | GGMD |
| 107 | NO: 14) | NO: 19) | (SEQ ID NO: 26) |
| NIH- | TLDYYAIG | CISNSGGSTY | VPSTYYSGSYYYTCHP |
| CoVnb- | (SEQ ID | (SEQ ID | GGMD |
| 108 | NO: 14) | NO: 20) | (SEQ ID NO: 26) |
| NIH- | TLDYYAIG | CITNSDGSTY | FPSTYYSGSYYYTCHP |
| CoVnb- | (SEQ ID | (SEQ ID | GGMD |
| 109 | NO: 14) | NO: 21) | (SEQ ID NO: 27) |
| NIH- | TLDYYAIG | CISSSDGSTY | ALSEGGYTIDGSSWCY |
| CoVnb- | (SEQ ID | (SEQ ID | HSVYGMD |
| 110 | NO: 14) | NO: 16) | (SEQ ID NO: 28) |
| NIH- | TLDYYAIG | CISSSDGSTY | VPSTYYSGTYYYNCHP |
| CoVnb- | (SEQ ID | (SEQ ID | GAMH |
| 111 | NO: 14) | NO: 16) | (SEQ ID NO: 29) |
| NIH- | TLDYYAIG | CISSSDGSTY | VPSTYYSGTYYYTCHP |
| CoVnb- | (SEQ ID | (SEQ ID | GGMD |
| 112 | NO: 14) | NO: 16) | (SEQ ID NO: 30) |
| NIH- | TLDYYAIG | CISSSDGSTY | VPSTYYSGTYYYTCHP |
| CoVnb- | (SEQ ID | (SEQ ID | GGMD |
| 113 | NO: 14) | NO: 16) | (SEQ ID NO: 30) |

The invention will be further described in the following examples, which do not limit the scope of the invention described in the claims.

### EXAMPLES

The Examples that follow are illustrative of specific embodiments of the disclosure, and various uses thereof. They are set forth for explanatory purposes only, and should not be construed as limiting the scope of the invention in any way.

### Example 1: Materials and Methods

### Immunization of Llamas

An adult male 16-year-old llama was immunized under contract at Triple J Farms (Kent Laboratories, Bellingham, Wash.). Subcutaneous injection was performed at multiple locations with 100 μg of SARS-Cov-2 S1 protein (S1N-05255, ACRO Biosystems) emulsified in complete Freund's adjuvant on day 0, followed by additional 100 μg immunizations emulsified with incomplete Freund's adjuvant on days 7, 14, 21, and 28. On day 35, peripheral blood was isolated and shipped on ice for further processing. Triple J Farms operates under established National Institutes of Health Office of Laboratory Animal Welfare Assurance certification number A4335-01 and United States Department of Agriculture registration number 91-R-0054.

### Generation of Immune Single-Domain Antibody Phage Display Library

The general synthesis methods utilized were adapted from work by Pardon et al., Nat Protoc. 2014; 9(3):674-93. Epub 2014/03/01. doi: PubMed PMID: 24577359; PubMed Central PMCID: PMCPMC4297639. Peripheral blood mononuclear cells (PBMCs) were isolated from llama whole blood using Uni-Sep Maxi density gradient tubes (#U-10, Novamed) according to the manufacturer's directions. The platelet-rich plasma was further processed and stored at −80° C. for use in measuring antibody titer. The PBMCs were rinsed with 1× phosphate buffered saline (PBS), aliquoted, and frozen at −80° C. prior to use. For total RNA isolation a minimum of 1×10⁷ PBMCs were processed using a RNeasy Mini Kit (Qiagen). First-strand synthesis of complimentary DNA from the resulting total RNA was prepared using the SuperScript™ IV First-Strand Synthesis kit (#18091050, Invitrogen) with random hexamer primed conditions and 2 μg total RNA. Synthesized first-strand complimentary DNA was used to amplify the heavy-chain variable domains using Q5 high-fidelity DNA polymerase (New England Biolabs) and the described primers (CALL001: 5′-GTCCTGGCTGCTCTTCTACAAGG-3′ (SEQ ID NO:34) and CALL002: 5′-GGTACGTGCTGTTGAACTGTTCC-3′ (SEQ ID NO:35)). The resulting amplified variable domains were separated on a 1.2% (w/v) low melting point agarose gel and the approximately 700 base pair band corresponding to the heavy chain only immunoglobulin was extracted and purified using QIAquick Gel Extraction Kit (Qiagen). A secondary amplification of the VHH domain was performed using modified primers (VHH-Esp-For: 5′-CCGGCCATGGCTGATGTGCAGCTGCAGGAGTCTGGRGGAGG-3′ (SEQ ID NO:36) and VHH-Esp-Rev: 5′-GTGCGGCCGCTGAGGAGACGGTGACCTGGGT-3′ (SEQ ID NO:37)) based on those used by Pardon et al. to introduce cloning compatible sequence for the phagemid pHEN2 (Pardon et al., Nat Protoc. 2014; 9(3):674-93. Epub 2014/03/01. PubMed PMID: 24577359; PubMed Central PMCID: PMCPMC4297639). The pHEN2 phagemid allowed for in-frame cloning of the VHH sequences with the pIII M13 bacteriophage gene and the inclusion of a C-terminal 6×His tag and triple myc tag separated from the pIII sequence by an amber stop codon (TAG). The amplified VHH sequences were then restriction endonuclease digested using NcoI and NotI (New England Biolabs) and purified from the reaction components. The resulting digested VHH sequences were ligated into NcoI/NotI digested pHEN2 phagemid at a 3:1 (insert:phagemid) ratio overnight at 16° C. followed by purification and then electroporation into phage-display competent TG-1 cells (#60502-1, Lucigen). The library was plated onto 2×YT agar plates containing 100 μg/mL carbenicillin and 2% glucose at 37° C. overnight. The resulting library contained >10⁸ independent clones. The library was pooled and archived as glycerol stocks. Standard phage amplification of the representative library was performed using M13KO7 helper phage (#18311019, Invitrogen) followed by precipitation with 20% polyethylene glycol 6000/2.5M sodium chloride on ice to purify the phage particles for downstream immunopanning. All post-reaction purifications utilized the MinElute PCR Purification Kit (Qiagen).

### Competitive Immunopanning

Selection of nanobodies that block the RBD-ACE2 interaction was performed using a bias-selection strategy. Standard radioimmunoassay tubes were coated with 500 μL of human ACE2 protein (#AC2-H52H8, ACRO Biosystems) solution at 5 μg/mL in sodium carbonate buffer, pH 9.6 overnight at 4° C. The coating solution was removed, and the tube blocked with a 2% (w/v) non-specific blocking solution which was alternated (bovine serum albumin, nonfat dry milk, or Li-Cor Intercept) each panning round to reduce enrichment of off-target clones. Amplified phage library (approximately 10¹¹ phage) was mixed 1:1 with the relevant blocking solution to yield a 500 μL volume to which 1 μg biotinylated RBD (#SPD-082E9) was added and allowed to associate for 30 minutes at room temperature with mixing at 600 rpm. The phage library complexed with biotinylated-RBD was then transferred to the blocked radioimmunoassay tube and allowed to bind for 30 minutes at room temperature with mixing at 600 rpm. The non-associated biotinylated-RBD was then recovered by addition of 10 μL Dynabeads™ M-270 Streptavidin for 10 minutes and the supernatant transferred to a new blocked-microcentrifuge tube. The magnetics beads were washed 10 times with 1×PBS and the bound phage eluted with 100 mM triethylamine solution. The resulting phage elution was used to infect TG-1 cells and additional phage amplification and immunoselections performed.

### Phage Display Clone Screening

Individual colonies were selected from the phage display enrichment plates and cultured in 2×YT-carbenicillin containing microbial media in a 96-deep well block at 37° C. with 300 rpm shaking for 4-6 hours. Expression of nanobody was induced by addition of isopropyl-beta-D-thiogalactoside (GoldBio) to a final concentration of 1 mM and continued incubation overnight. The cells were pelleted by centrifugation at 1,000×G for 20 minutes. The media supernatant was removed, and the block placed at −80° C. for 1 hour. The block was then allowed to equilibrate to room temperature for 15 minutes, 500 μL 1×PBS was added, and the block agitated at 1,500 rpm for 1 hour to allow for release of nanobody from the periplasmic space. The block was then centrifuged for 20 minutes at 2,000×G. Nunc Maxisorp plates were coated with SARS-CoV-2 RBD (SPD-052H3, ACRO Biosytems) as described above and blocked with 2% bovine serum albumin (BSA). Nanobody containing supernatant was transferred to the blocked plate and incubated for 1 hour at room temperature. The assay plate was washed and 100 μL peroxidase conjugated goat anti-alpaca VHH domain specific antibody (#128-035-232, Jackson ImmunoResearch) was transferred into each well and incubated for 1 hour at room temperature. After a final wash the plate was developed by addition of 100 μL tetramethylbenzidine (TMB) (#T5569, Sigma-Aldrich). Assay development was stopped by addition of 50 μL 1M sulfuric acid and the assay absorbance measured at 450 nm on a Biotek Synergy 2 plate reader. The assay plate was washed with 1×PBS 5 times between each step of the assay. Clones were considered positive if the optical density 450 nm was greater than two standard deviations above the background.

### Bio-Layer Interferometry Selection of Receptor Binding Domain Binding VHH Clones

ELISA-positive clones were further assessed by bio-layer interferometry binding to RBD. Assay buffer was defined as 0.1% BSA (w/v) in 1×PBS. All assay conditions were prepared in a Greiner 96-well plate (#655209) in a volume of 300 μL. Biotinylated SARS-CoV-2 S protein RBD (SPD-C82E9, ACRO Biosystems) with a C-terminal AviTag was used to ensure uniform directionality of the protein. Biotinylated-RBD was diluted into assay buffer at 1 μg/mL and immobilized onto streptavidin coated biosensors (#18-5019, BioForte) to a minimum response value of 1 nm on the Octet Red96 System (ForteBio). A baseline response was established in assay buffer prior to each association. Candidate clone supernatant prepared previously was diluted 1:1 with assay buffer and allowed to associate for 60 s and dissociate for 60 s, which provided a sufficient interval to detect positive binding. Biosensors were regenerated in 250 mM imidazole in assay buffer for 45 s which removed residual bound candidate nanobody and returned to the baseline well. This method allowed for detection of clones that bind RBD in both ELISA and a qualitative estimate of binding based on response curve dynamics. Clones positive on both the RBD ELISA and bio-layer interferometry measurement were selected for sequencing and further characterization.

### ProteOn XPR36 Surface Plasmon Resonance Affinity Measurement of NIH-CoVnb-112 RBD Binding

Surface plasmon resonance affinity and kinetic measurements were performed using the ProteOn XPR36 (Bio-Rad). Lanes of a general layer compact (GLC) chip were individually coated with 2 μg/mL SARS-CoV-2 Receptor Binding Domain (RBD) (#SPD-052H3, ACRO Biosystems) in 10 mM sodium phosphate pH 6.0 and attached to the chip following the standard 1-ethyl-3-(3-dimethylaminopropyl)carbodiimide hydrochloride (EDC)/N-hydroxysulfosuccinimide (sulfo-NHS) coupling chemistry available from the manufacturer resulting ˜1000 RU of protein deposited. Binding kinetics of NIH-CoVnb-112 were tested at 25° C. by flowing six concentrations varying from 300 to 0 nM at 100 μL/min for 90 s or more then dissociation was monitored for at least 600 s. Following each run, the chip was regenerated by flowing 0.85% phosphoric acid (˜pH 3.0) across the surface. Data analysis was performed with ProteOn Manager 2.1 software, corrected by subtraction of an uncoated column as well as interspot correction. The standard error of the fits was less than 10%. Binding constants were determined using the Langmuir model built into the analysis software.

### Sanger Dideoxy Sequencing

Clones enriched by phage display and positively selected by ELISA assay and bio-layer interferometry measurement were sequenced using a universal Lac-promoter primer (Lac-fwd: 5′-CGTATGTTGTGTGGAATTGTGAGC-3′ (SEQ ID NO:38)) with standard Sanger dideoxy sequencing at Genewiz. The resulting sequences with Quality Scores above 40 and with Contiguous Read Lengths of greater than 500 were considered reliable. Sequences were trimmed to include only the VHH coding region and the protein coding sequences aligned using the Clustal Omega algorithm (Sievers, et al., Mol Syst Biol. 2011; 7:539. Epub 2011/10/13. doi: PubMed PMID: 21988835; PubMed Central PMCID: PMCPMC3261699) included in SnapGene software (GSL Biotech LLC).

### Nanobody Expression and Purification

Lead candidates were expressed by transferring the phagemid from TG-1 cells into the BL21 (DE3) competent E. coli strain (C2527I, New England BioLabs). Cultures were grown in 250 mL 4×YT-carbenicillin in a 1-liter baffled flask at 37° C. and 300 rpm until the OD600 reached between 0.7-0.9. Protein expression was induced by addition of isopropyl-beta-D-thiogalactoside to a final concentration of 1 mM and the culture conditions reduced to 30° C. and 180 rpm for overnight growth. The expression cultures were pelleted at 8000×G for 10 minutes at 4° C.; the resulting pellets maintained on ice. Osmotic shock was achieved by resuspension of the cell pellet in TES buffer (0.2M Tris, 65 μM EDTA, 0.5M sucrose, pH 8.0) at ⅕th the original culture volume (e.g. 20 mL TES for 100 mL culture volume) and incubated on ice for 1 hour with occasional agitation. The suspension was pelleted again as described above and then resuspended in an equal volume of ice-cold 10 mM magnesium chloride and incubated on ice for 30 minutes with occasional agitation. A final centrifugation was performed at 17,000×G for 10 minutes at 4° C. to pellet all cellular debris. The resulting supernatant was filtered through a 0.22 μm syringe filter to remove any residual particulates.

Purification of the nanobody was accomplished by injection of the clarified osmotic shock supernatant onto a 1 mL HisTrap FF column (#17531901, Cytiva Lifesciences) attached to an AKTA Pure FPLC system. Unbound protein was washed with 10 column volumes 1×PBS followed by 10 column volumes 20 mM imidazole in 1×PBS. The enriched nanobody was eluted with 250 mM imidazole in 1×PBS. An automated collection of the unbound flow-through and fractionation of the wash and elution steps was achieved with the fraction collector F9-C. The nanobody typically eluted into two 1.5 mL fractions which were pooled and concentrated to a volume of approximately 1 mL using a 10 kDa molecular weight cut-off Centricon centrifugal concentrator (#MPE010025, EMD Millipore). The concentrated volume was then injected onto a Superdex 75 10/300 GL size exclusion chromatography column on the AKTA Pure system with 1×PBS as the eluent and fractions collected. This polishing step typically achieved a >90% purity as determined by SDS-PAGE. Protein concentration was estimated using absorbance at 280 nm and quantified using a standard Bicinchoninic acid protein assay (#23225, ThermoScientific) with BSA as the concentration standard.

### Bio-Layer Interferometry Affinity Measurement of RBD Binding VHH Clones

Bio-layer interferometry was used to measure the affinity binding constants of purified VHH clones. Assay buffer is defined as 0.1% BSA (w/v) in 1×PBS. All assay conditions were prepared in a Greiner 96-well plate (#655209) in a volume of 300 μL. Biotinylated SARS-CoV-2 S protein RBD (SPD-C82E9, ACRO Biosystems) with a C-terminal AviTag was used to ensure uniform directionality of the protein. Biotinylated-RBD was diluted into assay buffer at 1 μg/mL and immobilized onto streptavidin coated biosensors (#18-5019, BioForte) to a minimum response value of 1 nm on the Octet Red96 System (ForteBio). A baseline response was established in assay buffer prior to each association. The purified VHH clones were diluted into assay buffer at the specified concentrations (typically 1,000 nM to 0 nM). The VHH clones were allowed to associate for 180-240 s followed by dissociation for 300-600 s in the same baseline wells. The assay included one biosensor with only assay buffer which was used as the background normalization control. Using the ForteBio Data Analysis suite, the data was normalized to the association curves following background normalization and Savitzky-Golay filtering. Curve fitting was applied using global fitting of the sensor data and a steady state analysis calculated to determine the association and dissociation constants.

### Receptor Binding Domain Competition Assay

SARS-Cov-2 RBD (#SPD-052H3, ACRO Biosystems) was coated at 0.5 μg/mL in sodium carbonate buffer (pH 9.6), 100 μL per well, onto Nunc Maxisorp plates overnight at 4° C. Coating solution was removed, and plate blocked with 300 μL 2% BSA (#5217, Tocris) in 1×PBS for 1 hour at room temperature. Purified nanobodies were diluted at specified concentrations in 0.2% BSA in 1×PBS and transferred to the blocked plate in triplicate. The nanobodies were incubated for 45 minutes to allow for association with RBD. Biotinylated ACE2 (AC2-H82E6, ACRO Biosystems) was prepared at 0.2 μg/mL in 0.2% BSA solution. 10 μL of the ACE2-biotin solution was transferred into each well of the assay and allowed to incubate for 15 minutes with at 600 rpm. The assay plate was washed and 100 μL of poly-streptavidin (#85R-200, Fitzgerald Industries International) diluted 1:2000 in 2% BSA solution was transferred to each well and incubated with at 600 rpm for 30 minutes. After a final wash the plate was developed by addition of 100 μL tetramethylbenzidine (#T5569, Sigma-Aldrich). Assay development was stopped by addition of 50 μL 1M sulfuric acid and the assay absorbance measured at 450 nm on a Biotek Synergy 2 plate reader. The assay plate was washed with 1×PBS 5 times between each step of the assay.

### Receptor Binding Domain Variant Competition Assay

SARS-CoV-2 RBD variants have been noted which increase the affinity for human ACE2 binding. Competition between the RBD mutants was performed as described above with the novel mutation RBD proteins replacing the canonical RBD sequence. SARS-CoV-2 RBD mutants W436R, N354D/D364Y, and V367F (#SPD-552H7, SPD-S52H3, and SPD-S52H4 respectively, ACRO Biosystems) were coated at 0.5 μg/mL in sodium carbonate buffer (pH9.6), 100 μL per well, onto Nunc Maxisorp plates overnight at 4° C. The assay was completed as described above.

### Genscript SARS-CoV-2 Neutralization Antibody Detection Kit

Secondary assessment of the RBD-ACE2 interaction blocking potential of the isolated nanobodies was performed using the Genscript SARS-Cov-2 Neutralization Antibody Detection Kit (#L00847, Genscript) according to the manufacturer's instructions. Briefly, horseradish peroxidase conjugated RBD was diluted using the supplied assay buffer and then incubated with specified concentrations of nanobody for 30 minutes at 37° C. The samples were then transferred onto the supplied human ACE2-coated assay plate and incubated for 15 minutes at 37° C. The plate was washed using the supplied solution and the assay developed using a supplied 3,3′,5,5′-Tetramethylbenzidine reagent. The assay was stopped with 50 μL of the supplied Stop Solution. The assay was measured at 450 nm on a Biotek Synergy 2 plate reader.

### SARS-CoV-2 Pseudotyped Lentivirus-Based Transduction Fluorescence Inhibition Assay

All lentiviruses were propagated in HEK293T/17 cells (ATCC #CRL-11268) according to published Current Protocols in Neuroscience41. Briefly, 293 T cells were transiently transfected with plasmids expressing SARSCoV-2 spike protein (GenScript MC_0101081, human codon optimized, ER retention signal removed), psPAX2 (Addgene #12260), and a lentiviral transfer vector CD512-EF1a-RFP (System Biosciences CD512B-1) using Lipofectamine 2000. Supernatant was collected 48 h post transfection and concentrated by centrifugation at 50,000×g for 2 h over a 20% sucrose cushion. Pellets were resuspended in PBS and used for infection. All titers were determined by performing biological titration of fluorescent viruses by flow cytometry.

For transduction assays, HEK293 Ts expressing human Angiotensin-Converting Enzyme 2 (HEK293T-ACE2, BEI Resources, NR-52511) were plated at the density of 50,000 cells/well in a 6-well plate. Cells were transduced with SARS-CoV-2 pseudotyped recombinant lentiviruses expressing RFP (S-CD512-EF1a-RFP) with multiplicity of infection (MOI) of 0.5+/−nanobodies at described concentrations. Media on cells was replaced the next day. 48 h post transduction, cells were released from wells with trypsin and fixed in 1% formaldehyde. BD LSRFortess Flow Cytometer was used to determine percent fluorescent cells and the mean fluorescent intensity per sample. All experiments were performed in triplicate.

Expression of NIH-CoVnb-112 in Pichia pastoris

Increased expression yield and elimination of potential endotoxin from the bioprocess was achieved by cloning and expression of the nanobody in the methylotrophic yeast Pichia pastoris. The EasySelect Pichia Expression Kit (#K1740-01, Invitrogen) was used for cloning of the VHH sequence into expression vector pICZa by amplification of the pHEN2 containing phagemid with reformatting primers (Pichia-nominal-fwd.: 5′-TAT CTC TCG AGA AAA GAG ATG TGC AGC TGC AGG AGT CTG-3′ (SEQ ID NO:39) and Pichia-nominal-rev: 5′-TTG TTC TAG ATT AGT GAT GGT GAT GTG CGG CCGC-3′ (SEQ ID NO:40)). The reformatting primers introduced XhoI and XbaI (New England Biolabs) restriction endonuclease sites which allowed for in-frame cloning of the VHH sequence with the α-factor secretion signal and includes a vector independent 6×His tag at the C-terminus. The resulting expression vector was linearized using Scal-HF (New 1 M sorbitol and 100 μg/mL Zeocin. Resulting clones were confirmed for recombination and for phenotype by small scale expression. For scaled expression, selected clones were grown in buffered glycerol complex medium to an OD600 of 1 prior to addition of methanol to a final concentration of 0.5%. The culture was batch-fed every 24 h to a final methanol concentration of 0.5% until harvest of the culture supernatant. Spent cells were removed by centrifugation at 3000×G for 10 min at room temperature and the supernatant clarified by filtration through a 0.45 μm filtration unit. The media supernatant was concentrated using a Minimate tangential flow filtration system (#OAPMPUNV, Pall) and buffer exchanged using 1×PBS containing 10 mM imidazole. Purification was performed as described above using Ni-NTA affinity chromatography followed by polishing on a Superdex 75Increase 10/300 GL size exclusion column.

### Nebulization Stability Assessment of NIH-CoVnb-112

Stability following nebulization of Pichia pastoris expressed NIH-CoVnb-112 was performed using an Aerogen Solo High-Performance Vibrating Mesh nebulizer placed in line with a custom glass bead condenser. A plastic culture tube was fitted with a glass-pore frit and filled with sterilized 5 mm borosilicate glass beads. A three-way stopcock was positioned distal to the frit to prevent pressurization during nebulization. A 2 mg/mL SEC polished NIH-CoVnb-112 solution was prepared in 0.9% normal saline to model potential patient delivery. The nanobody was nebulized and the resulting condensate incubated at 37° C. for 24 hr to mimic exposure to body temperature. The nebulized, 37° C. treated nanobody was then collected for stability assessments and protein concentration measurements by BCA assay. Equal masses of pre and post-nebulization samples were denatured in LDS sample buffer (Invitrogen) and run on a NuPAGE 12% Bis-Tris precast polyacrylamide gel with SeeBlue Plue 2 protein standards. Additional pre and post-nebulization samples were injected onto a Superdex 75 Increase 10/300 GL size exclusion column operating on an AKTA Pure 25 M system.

### Stability of NIH-CoVnb-112 in Human Plasma

NIH-CoVnb-112 expressed in Pichia pastoris was diluted from a concentrated stock solution into apheresis derived pooled human plasma (#IPLA-N, Innovative Research) to a final concentration of 5 μM and incubated at 37° C. for either 24 hr or 48 hr with gentle rotation. An identical sample set was prepared at 5 μM in a solution containing 35 mg/mL recombinant human albumin (#A9731, Sigma-Aldrich) and incubated at 37° C. for either 24 hr or 48 hr with gentle rotation. A no-incubation control for each the plasma and recombinant human albumin conditions was prepared at 5 μM. The samples were prepared in a manner providing all conditions complete at the same time. The samples were diluted 1:10 with 1×PBS to yield a final nanobody concentration of 500 nM and Bio-layer Interferometry was performed using immobilized biotinylated SARS-CoV-2 S protein RBD to determine retention of binding potential.

### Determining Melting Temperature and Refolding by Circular Dichroism

Circular Dichroism (CD) was performed using a Jasco J-815 Spectropolarimeter. For thermal stability measurements NIH-CoVnb-112 was diluted to 10 μg/mL in deionized water and placed in a quartz cuvette with 1 cm path length and CD was measured at an ultraviolet wavelength of 205 nm. NIH-CoVnb-112 heated from 25° C. to 85° C. at a rate of 2.5° C./min while stirring and then cooled back to 25° C. at the same rate.

### Example 2: Isolation, Purification and Characterization of High Affinity Nanobodies

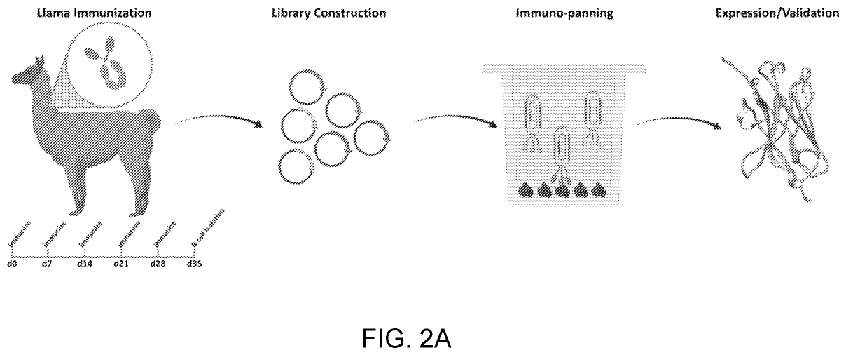

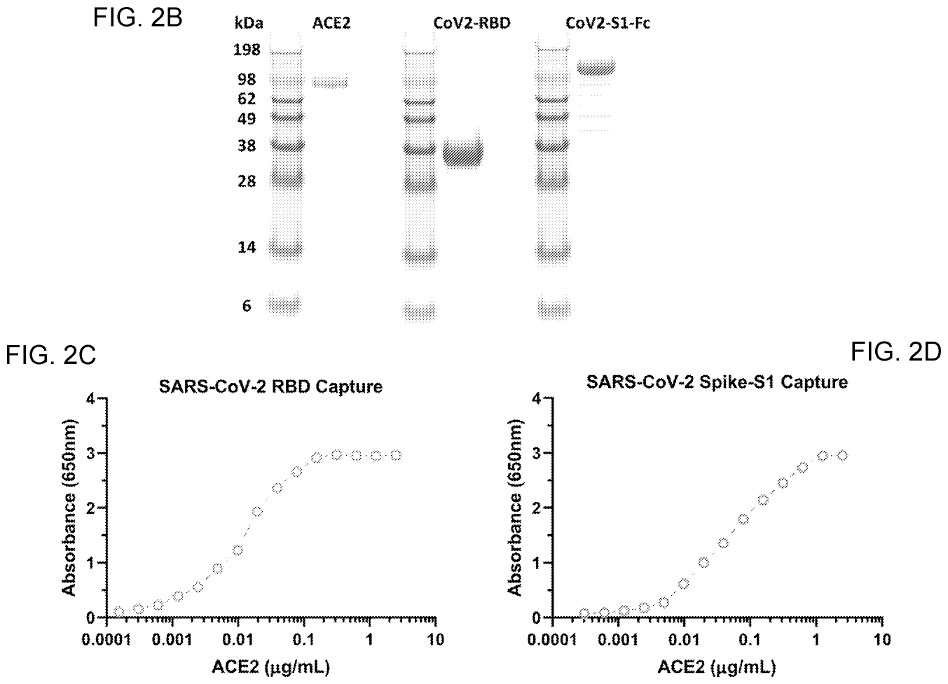

Using standard methods to immunize llama, B-cell nanobody DNA sequences were isolated and a construct phage display library with over 10⁸ clones was created (FIG. 2A). From this phage library, 13 unique lead candidate nanobodies that bind to the SARS-CoV-2 spike protein RBD were isolated, several of which block the spike protein-ACE2 interaction with high potency.

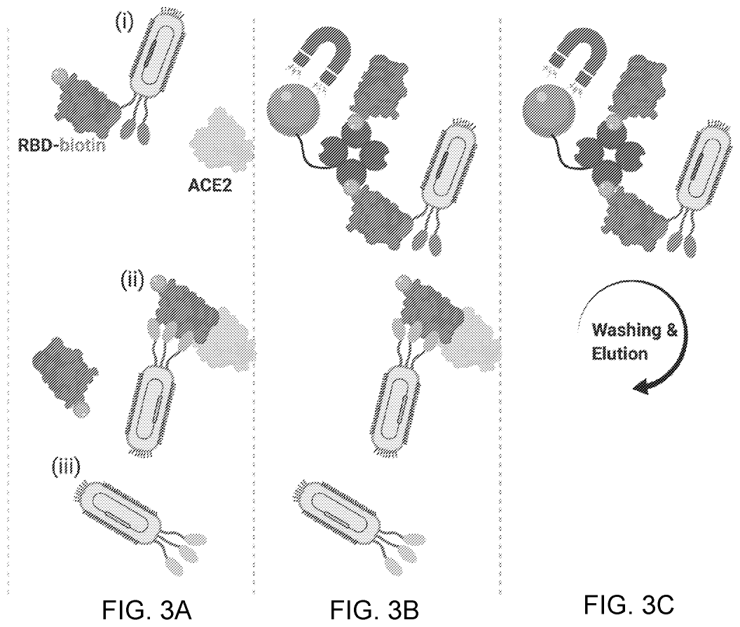

To isolate the candidate nanobodies, a novel screening strategy was designed and executed to specifically select for nanobodies that not only bound to the SARS-Cov-2 spike RBD, but also interfered with the interaction with the human ACE2 protein. The purity of in vitro binding of commercially available recombinant spike protein RBD and human ACE2 protein (FIGS. 2 B-D) that were used for the screening strategy was validated. In this screening strategy (FIG. 3), recombinant human ACE2 protein was immobilized in tubes. Then, biotinylated-RBD was incubated with the nanobody phage library and allowed to interact with the immobilized ACE2. Biotinylated-RBD with no associated nanobodies, or with nanobody associations that did not block the ACE2 binding domain, bound the immobilized ACE2. Biotinylated-RBD with associated nanobodies that blocked the ACE2 binding domain remained in solution and were recovered using streptavidin-coated magnetic particles that bind to the biotin. Nanobodies that did not bind to RBD were removed during washing of the magnetic beads. This method allowed for specific enrichment of functional nanobodies that bind to the RBD and compete for the RBD-ACE2 binding surface.

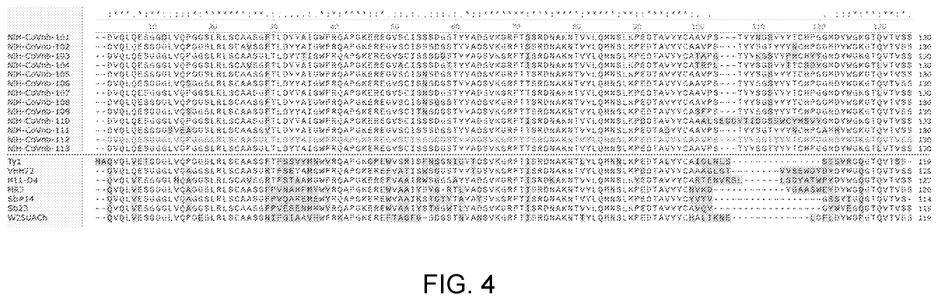

Using the novel screening strategy, hundreds of phage were isolated, which when sequenced, revealed 13 unique full length nanobody DNA sequences, termed NIH-CoVnb-101 through NIH-CoVnb-113 (FIG. 4). These sequences were distinct from the previously published sequences that also bind SARS-CoV-2 spike protein. Additional reports demonstrate compelling binding to SARS-CoV-2 spike protein, yet have not disclosed nanobody sequences allowing for a direct comparison. The complementarity determining region (CDR) 3 domain responsible for much of the specific binding of nanobodies to their targets, in NIH-CoVnb-112 and the other new nanobodies were generally longer than those currently reported. Twelve of the 13 nanobodies are relatively similar to each other, whereas one nanobody (NIH-CoVnb-110) was distinct from the other 12. These findings indicated that novel nanobody DNA sequences were isolated.

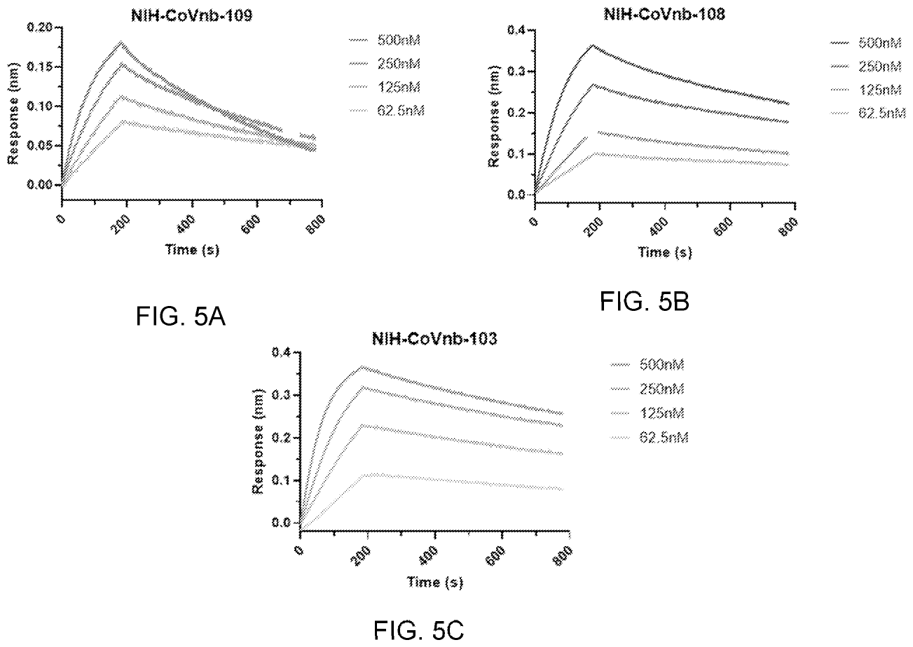

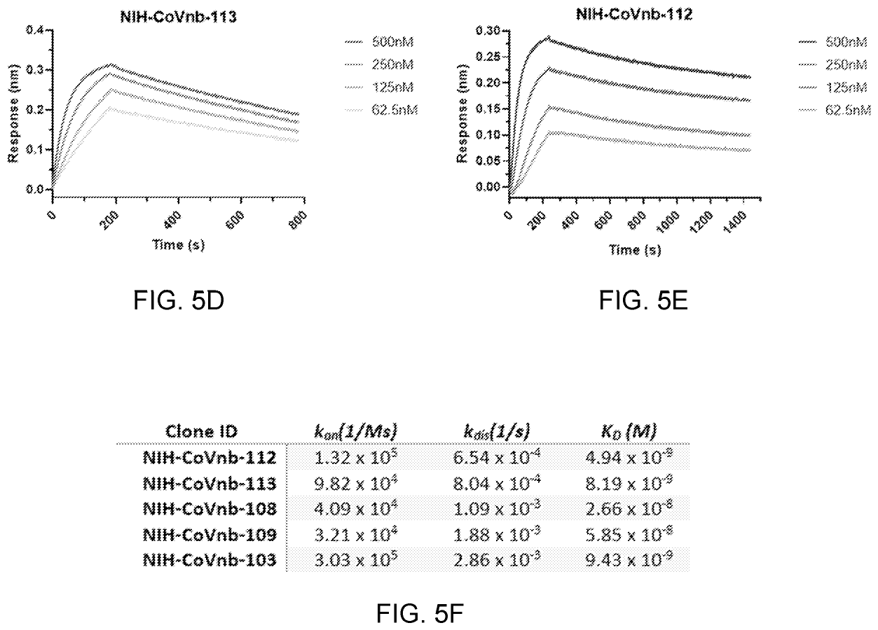

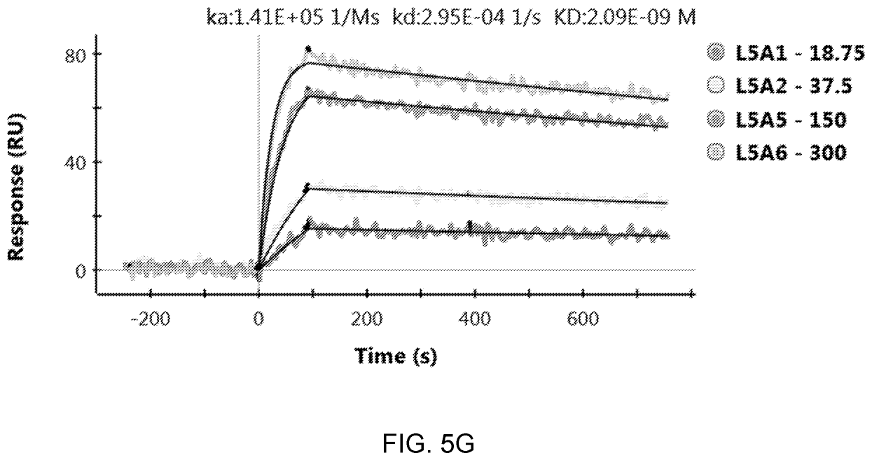

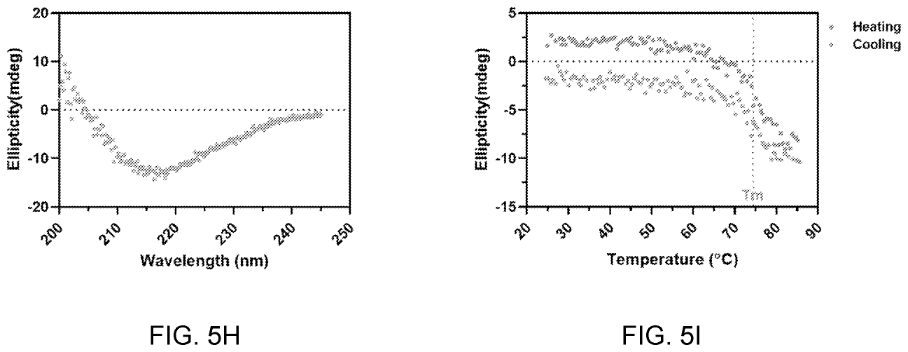

Representatives from each of these unique nanobody sequences were produced in bacteria, purified, and tested for binding to SARS-Cov-2 spike RBD (FIG. 5). All of the nanobodies bound to recombinant SARS-Cov-2 spike RBD with high affinity. The strongest binding nanobody was NIH-CoVnb-112 (FIG. 5E) with an affinity of 4.9 nM. NIH-CoVnb-112 had both a fast on rate (1.3e5/M/sec.) and a slow off rate (6.54e-4/sec.), with kinetics compatible with 1:1 binding. In a complementary measurement, ProteOn XPR36 surface plasmon resonance provided good agreement with an affinity of 2.1 nM (FIG. 5G). These results indicate that the novel nanobodies bind to the SARS-Cov-2 spike RBD with very high affinity. The long CDR3 in NIH-CoVnb-112 and the other new nanobodies can in part underlie their extraordinarily high affinity, though this is clearly not the only factor in that the nanobody with the longest CDR3, NIH-CoVnb-110, is not one of the top 5 highest affinity nanobodies. Measurements using circular dichroism (CD) during heating reveal that the nanobody structure resists unfolding until 74.4° C. (FIGS. 5H-5I) and upon cooling 73% of the structure returned to the baseline CD value. These data support an extremely stable, robust, high affinity nanobody.

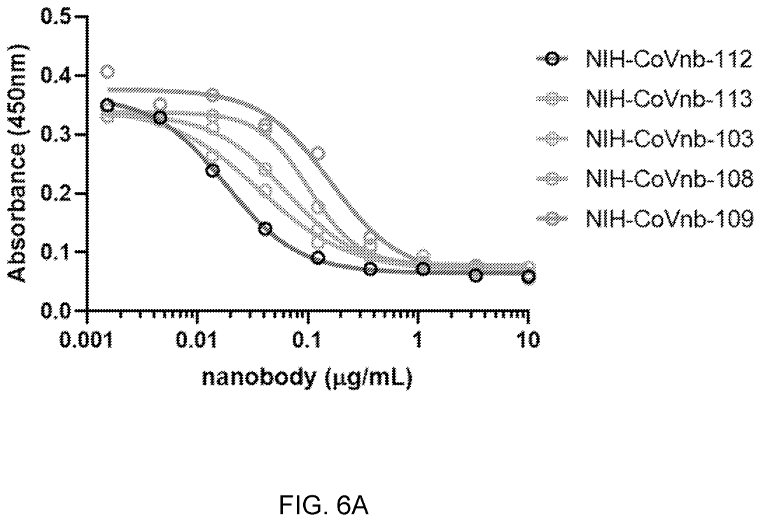

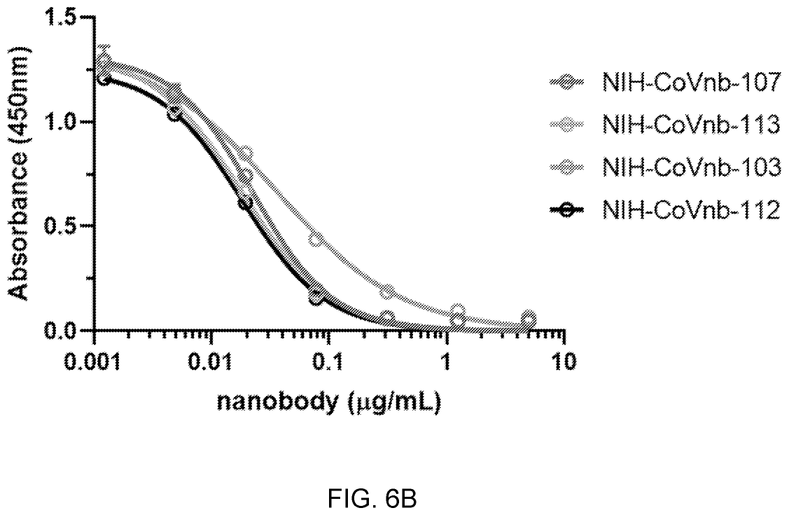

In an important validation of the in vitro efficacy of the candidate nanobodies, the nanobodies were able to interfere with SARS-Cov-2 spike RBD binding to human ACE2 protein (FIG. 6B). Specifically, a competitive inhibition assay was designed and implemented, in which recombinant RBD was coated onto Enzyme Linked Immuno-Sorbent Assay (ELISA) plates and soluble ACE2 binding was assessed. Without any interference, soluble ACE2 binding was indicated by high colorimetric absorbance. At increasing concentrations, each of the new nanobodies, showed progressively less ACE2 binding. For the most potent anti-SARS-CoV-2 RBD nanobody, NIH-CoVnb-112, the concentration at which 50% of the ACE2 binding was blocked (half maximal effective concentration; termed EC₅₀) was found to be 0.02 micrograms/mL, equivalent to 1.11 nM. The rank order of ACE2 competition EC₅₀ matched the rank order of RBD binding affinities for the novel nanobodies assessed. As an additional confirmation, the experiment was repeated using a commercially available SARS-Cov-2 spike RBD-human ACE2 protein competitive inhibition assay (GenScript). The results were very similar to those obtained using the initial assay (FIG. 6B). These results indicate that the novel nanobodies block SARS-Cov-2 spike protein RBD binding to ACE2, an essential human receptor responsible for viral infection.

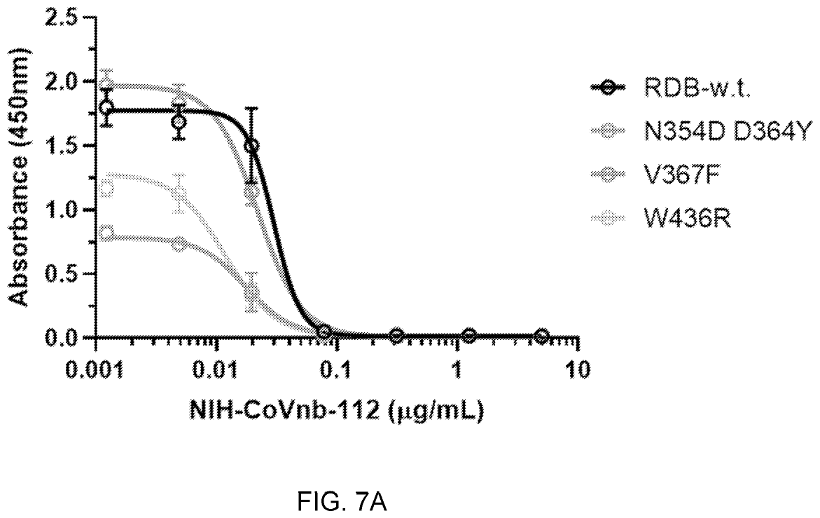

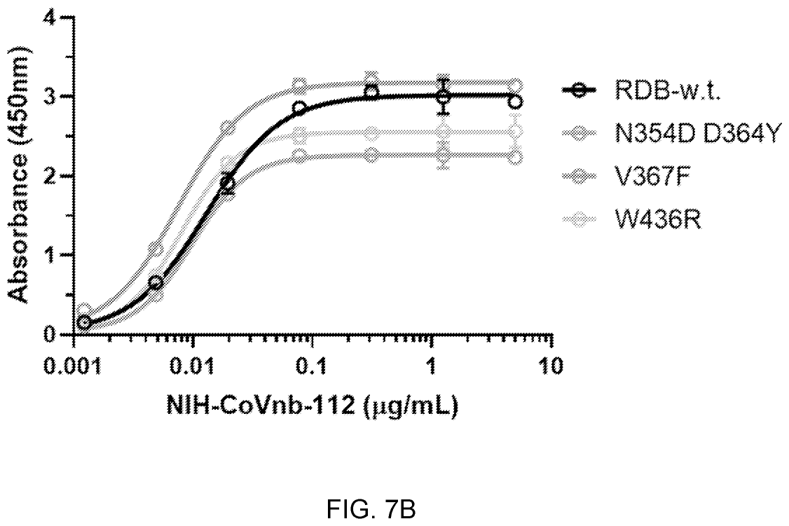

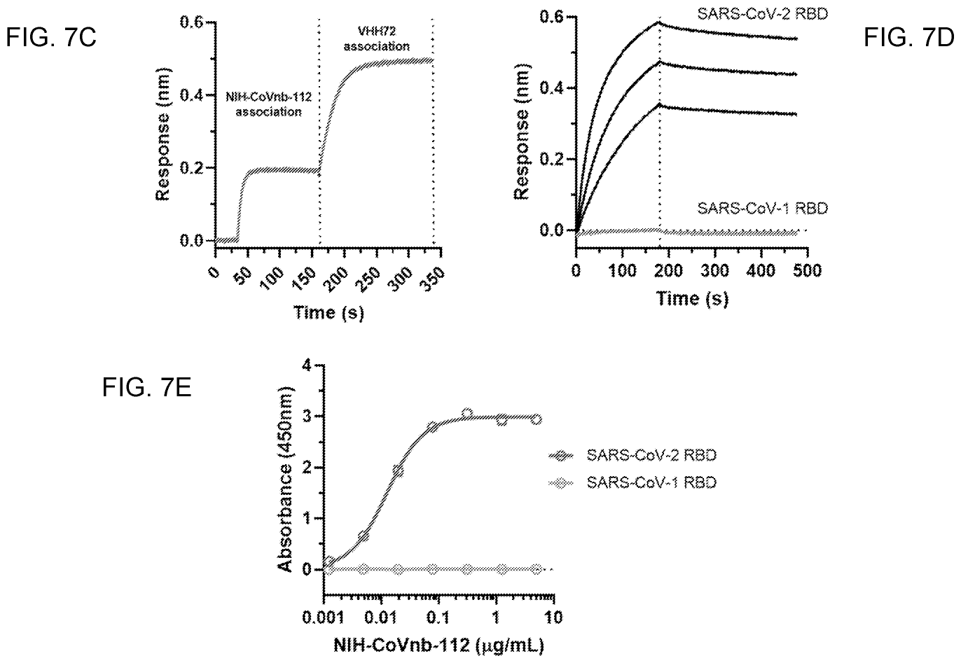

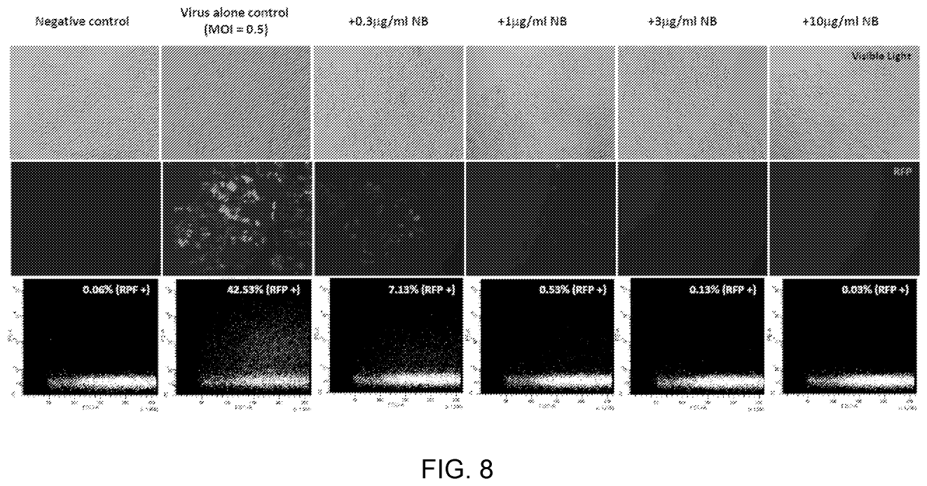

There have been many variants of the spike protein RBD described recently that increase the binding affinity to the human ACE2 receptor (Ou J, et al., bioRxiv 2020. Epub Apr. 20, 2020. doi. Several of these variants including N354D D364Y, V367F, and W436R have been reported to have up to 100 fold higher affinity for ACE2 than the prototype RBD in vitro. NIH-CoVnb-112 blocked interaction between human ACE2 and three of these variants with similar EC₅₀ compared to its blocking effects on the prototype sequence spike protein RBD (FIG. 7A). Binding affinity of NIH-CoVnb-112 to the variants was also similar to that of the prototype sequence (FIG. 7B).

Next, it was assessed whether NIH-CoVnb-112 binds to the same spike protein RBD epitope as the previously reported nanobody VHH7. If both nanobodies bind to the same epitope, their signals would occlude each other when applied at saturating concentrations on bilayer interferometry. In contrast, if they bind to different epitopes, their signals would be additive on bilayer interferometry. When NIH-CoVnb-112 was applied at 500 nM (>100× greater than the KD) for long enough to reach steady state, and then VHH72 was applied, the signals were clearly additive (FIG. 7C). This result clearly indicates that NIH-CoVnb-112 binds to a SARS-CoV-2 spike protein RBD epitope that is distinct from that of VHH72. This result is consistent with the reported findings that VHH72 recognizes a non-ACE2 binding motif on the spike protein24, whereas NIH-CoVnb-112 has RBD-ACE2 interaction disruption potency that is quantitatively similar to its affinity—a result that is most consistent with NIH-CoVnb-112 binding directly to the ACE2 interaction domain.

Additional tests were performed to determine if NIH-CoVnb-112 possesses the ability to also bind to the SARS-CoV-1 spike protein RBD domain. A biotinylated version of NIH-CoVnb-112 which could be used in parallel to test binding to several targets was prepared. Biotinylated NIH-CoVnb-112 was immobilized onto streptavidin Octet sensors and then allowed to associate (FIG. 7D) with either SARS-CoV-1 RBD or SARS-CoV-2 RBD at 500-125 nM concentrations. As previously demonstrated, NIH-CoVnb-112 binds robustly to SARS-CoV-2 RBD, yet there was essentially no binding to SARS-CoV-1 RBD. In an orthogonal confirmation, an ELISA was performed in which each RBD was coated onto a standard ELISA plate and then allowed to incubate with NIH-CoVnb-112. The ELISA results were similar to the Octet results (FIG. 7E). NIH-CoVnb-112 bound strongly to SARS-CoV-2 RBD and did not associate with SARSCoV-1 RBD. These results clearly indicate that NIH-CoVnb-112 does not cross react and bind to SARS-CoV-1 spike protein RBD.

The presence of bacterial endotoxins introduces an unwanted liability during the production of NIHCoVnb-112 due to expression in E. coli. To remove this risk, the coding sequence for the nanobody was cloned into an expression vector for the methylotrophic yeast Pichia pastoris. The expression vector, pICZalpha, contains an alpha-factor secretion signal sequence which directs the expressed protein into the media supernatant. Following selection of a positive recombinant clone it was found an expression yield of 40 mg per liter following 96 h of methanol batch-fed culture.

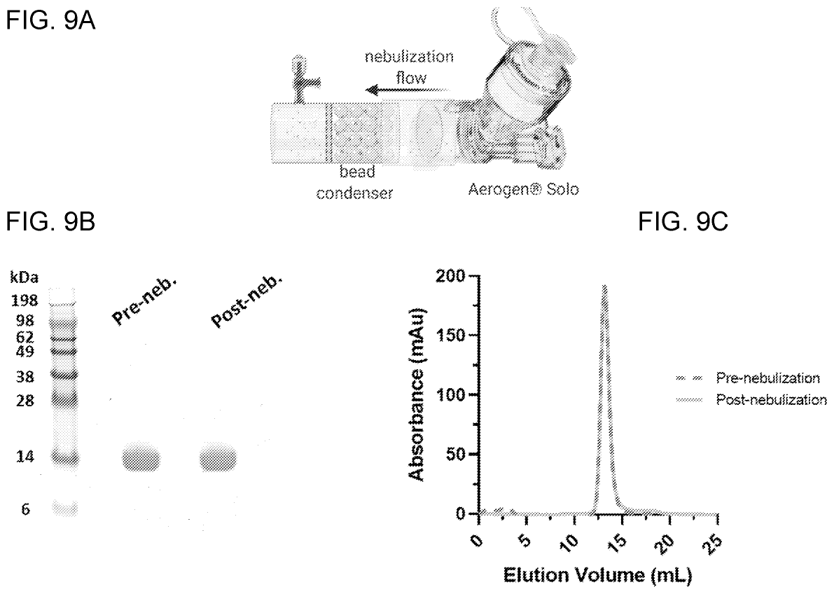

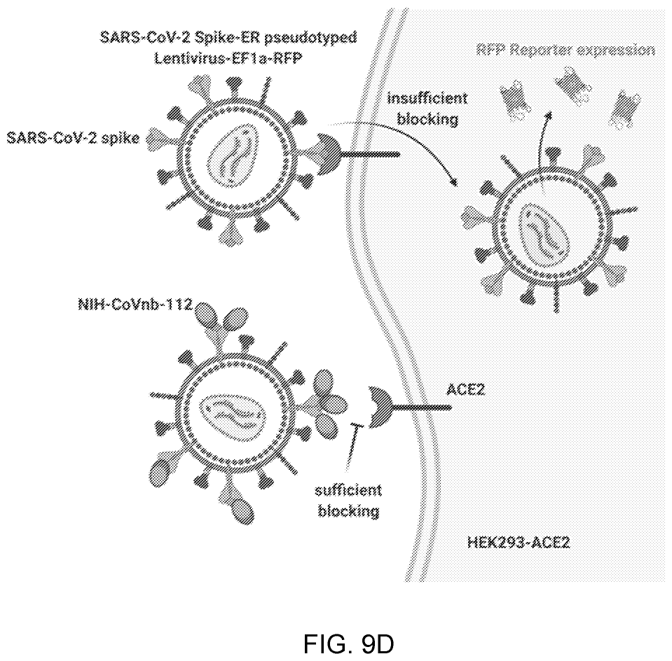

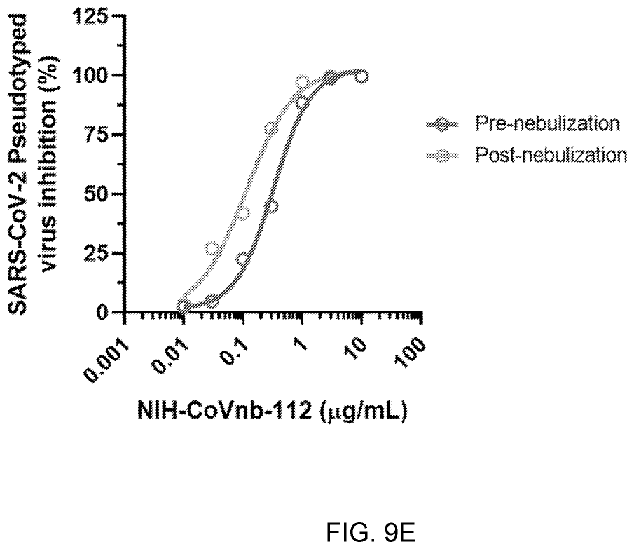

Using the commercially available Aerogen Solo vibrating mesh nebulizer, Pichia expressed NIH-CoVnb-112 was nebulized into an in line custom bead condenser (FIG. 9A) to collect the post-nebulized nanobody. Greater than 90% of the total input protein mass was recovered. Following nebulization, the recovery pre-nebulization and post-nebulization samples were incubated at 37° C. for 24 hr to provide physiologic temperature exposure.

When assessed by SDS-PAGE gel (FIG. 9A) the pre-nebulization and post-nebulization NIHCoVnb-112 appeared identical with no evidence of degradation or aggregation products. Similarly, analysis by size-exclusion chromatography (FIG. 9C) on a Superdex 75 column yielded similar elution profiles with no evidence of degradation or aggregation relative to the pre-nebulization sample. It appears that NIH-CoVnb-112 is resilient to degradation or aggregation during nebulization.

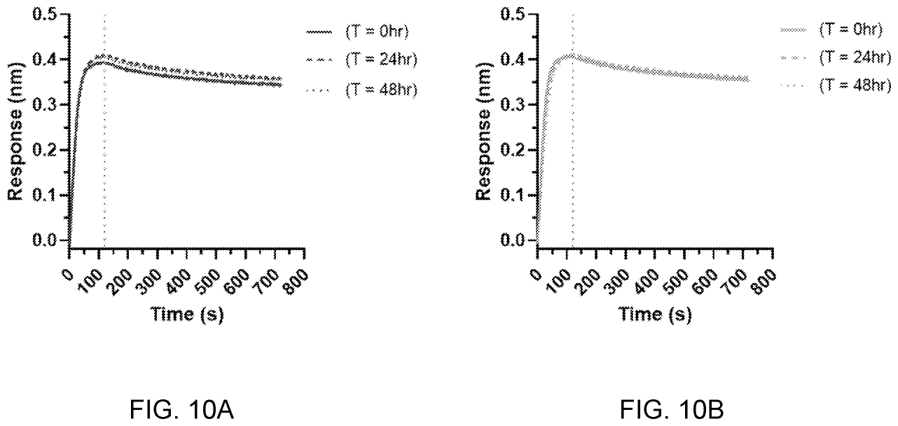

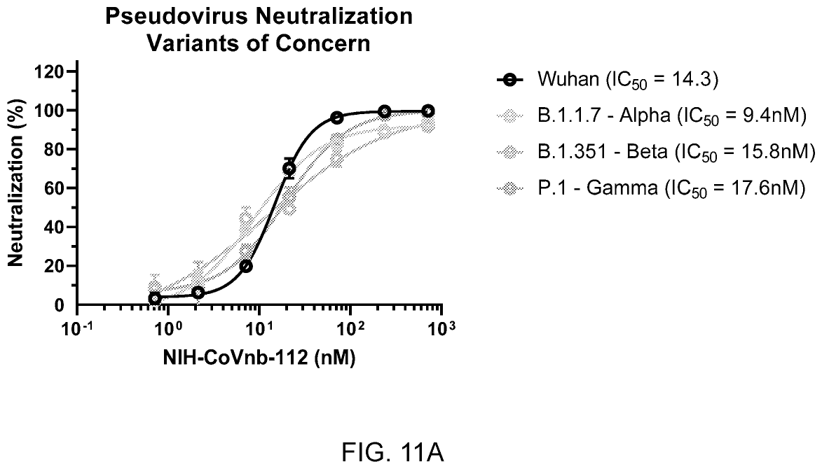

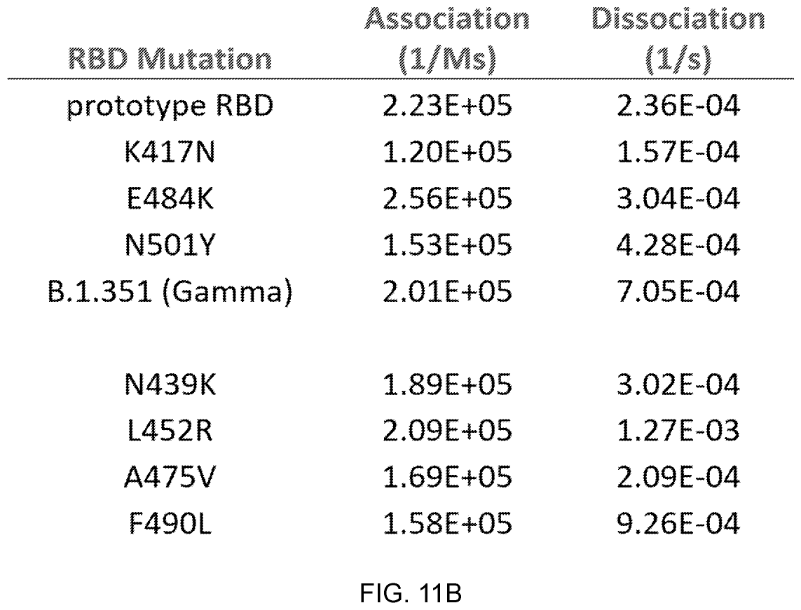

To further assess the stability of NIH-CoVnb-112, incubation in pooled normal human plasma and recombinant human albumin was performed followed by affinity measurement to assess preservation of SARS-CoV-2 RBD binding potential. NIH-CoVnb-112, at 5 μM, was incubated for 24 or 48 hrs in the presence of human plasma, which contains proteases, or recombinant human albumin alone at 37° C. with gentle mixing. In addition, for each condition a time zero control was prepared to account for potential matrix effects. The samples were diluted to 500 nM based on the NIH-CoVnb-112 starting concentration and assessed by biolayer interferometry for binding to SARS-CoV-2 RBD. NIH-CoVnb-112 binding (FIG. 10A) SARS-CoV-2 RBD following treatment in pooled human plasma at time zero, 24 hr, and 48 hr had negligible impact on binding. Similarly, NIH-CoVnb-112 binding (FIG. 10B) SARS-CoV-2 RBD following treatment in recombinant human albumin at all time points had no apparent effect on binding. While further in vivo characterization is necessary, these data support the interpretation that NIH-CoVnb-112 is satisfactorily stable in the presence of plasma.

Pseudotyped SARS-CoV-2 spike protein bearing lentivirus with an RFP reporter system was used to test if NIH-CoVnb-112 could inhibit the transduction of human embryonic kidney cells (HEK293T/17) overexpressing human ACE2 (HEK293-ACE2). Transduction of HEK293-ACE2 cells resulted in RFP expression (FIG. 9D) in the absence of an inhibitory agent. The pseudotyped virus were produced using a human codon optimized SARSCoV-2 spike protein sequence with the endoplasmic reticulum retention signal removed. Pseudotyped virus were purified over a sucrose cushion and viral titer determined using digital droplet PCR. A serial dilution of Pichia expressed, pre-nebulization and post-nebulization NIH-CoVnb-112 samples were incubated with a Multiplicity of Infection (MOI) of 0.5 followed by incubation on HEK293-ACE2 cells for 48 hrs. Following transduction, brightfield and epifluorescence images were acquired for both pre-nebulization and post-nebulization conditions. Incubation at or above 0.3 μg/mL NIH-CoVnb-112 resulted in robust inhibition for both conditions. Following trypsinization and fixation, the individual conditions were analyzed by flow cytometry to determine the percentage of RFP expressing cells. The population of single events were recorded for both pre-nebulization and post-nebulization conditions. The number of RFP positive events for each condition were normalized relative to the virus-alone control to yield a percent inhibition of the SARS-CoV-2 pseudotyped lentivirus (FIG. 9E). The pre-nebulization NIH-CoVnb-112 has an EC50 of 0.323 μg/mL (23.1 nM) while the post-nebulization NIH-CoVnb-112 has an EC50 of 0.116 μg/mL (8.3 nM). The difference in EC50 values between the two conditions is most likely an effect of assay variance and we do not speculate that nebulization produced an increase in potency. Thus, NIHCoVnb-112 potently inhibits viral transduction in an infection relevant pseudotyped SARS-CoV-2 virus model.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

### REFERENCES

1. Cui J, Li F, Shi Z L. Origin and evolution of pathogenic coronaviruses. Nat Rev Microbiol. 2019; 17(3):181-92. Epub 2018/12/12. doi: https://doi.org/10.1038/s41579-018-0118-9. PubMed PMID: 30531947; PubMed Central PMCID: PMCPMC7097006.
2. Zhou P, Yang X L, Wang X G, Hu B, Zhang L, Zhang W, et al. A pneumonia outbreak associated with a new coronavirus of probable bat origin. Nature. 2020; 579(7798):270-3. Epub 2020/02/06. doi: https://doi.org/10.1038/s41586-020-2012-7. PubMed PMID: 32015507; PubMed Central PMCID: PMCPMC7095418.
3. Huang C, Wang Y, Li X, Ren L, Zhao J, Hu Y, et al. Clinical features of patients infected with 2019 novel coronavirus in Wuhan, China. Lancet. 2020; 395(10223):497-506. Epub 2020/01/28. doi: https://doi.org/10.1016/S0140-6736(20)30183-5. PubMed PMID: 31986264.
4. Yuan M, Wu N C, Zhu X, Lee C D, So R T Y, Lv H, et al. A highly conserved cryptic epitope in the receptor-binding domains of SARS-CoV-2 and SARS-CoV. Science. 2020. Epub 2020/04/05. doi: https://doi.org/10.1126/science.abb7269. PubMed PMID: 32245784.
5. Li W, Moore M J, Vasilieva N, Sui J, Wong S K, Berne M A, et al. Angiotensin-converting enzyme 2 is a functional receptor for the SARS coronavirus. Nature. 2003; 426(6965):450-4. Epub 2003/12/04. doi: https://doi.org/10.1038/nature02145. PubMed PMID: 14647384; PubMed Central PMCID: PMCPMC7095016.
6. Li W, Zhang C, Sui J, Kuhn J H, Moore M J, Luo S, et al. Receptor and viral determinants of SARS-coronavirus adaptation to human ACE2. EMBO J. 2005; 24(8):1634-43. Epub 2005/03/26. doi: https://doi.org/10.1038/sj.emboj.7600640. PubMed PMID: 15791205; PubMed Central PMCID: PMCPMC1142572.
7. Shang J, Ye G, Shi K, Wan Y, Luo C, Aihara H, et al. Structural basis of receptor recognition by SARS-CoV-2. Nature. 2020; 581(7807):221-4. Epub 2020/04/01. doi: https://doi.org/10.1038/s41586-020-2179-y. PubMed PMID: 32225175; PubMed Central PMCID: PMCPMC7328981.
8. Yan R, Zhang Y, Li Y, Xia L, Guo Y, Zhou Q. Structural basis for the recognition of SARS-CoV-2 by full-length human ACE2. Science. 2020; 367(6485):1444-8. Epub 2020/03/07. doi: https://doi.org/10.1126/science.abb2762. PubMed PMID: 32132184; PubMed Central PMCID: PMCPMC7164635.
9. Descotes J. Immunotoxicity of monoclonal antibodies. MAbs. 2009; 1(2):104-11. Epub 2010/01/12. doi: https://doi.org/10.4161/mabs.1.2.7909. PubMed PMID: 20061816; PubMed Central PMCID: PMCPMC2725414.
10. Dumoulin M, Conrath K, Van Meirhaeghe A, Meersman F, Heremans K, Frenken L G, et al. Single-domain antibody fragments with high conformational stability. Protein Sci. 2002; 11(3):500-15. Epub 2002/02/16. doi: https://doi.org/10.1110/ps.34602. PubMed PMID: 11847273; PubMed Central PMCID: PMCPMC2373476.
11. Hamers-Casterman C, Atarhouch T, Muyldermans S, Robinson G, Hamers C, Songa E B, et al. Naturally occurring antibodies devoid of light chains. Nature. 1993; 363(6428):446-8. Epub 1993/06/03. doi: https://doi.org/10.1038/363446a0. PubMed PMID: 8502296.
12. Muyldermans S. Nanobodies: natural single-domain antibodies. Annu Rev Biochem. 2013; 82:775-97. Epub 2013/03/19. doi: https://doi.org/10.1146/annurev-biochem-063011-092449. PubMed PMID: 23495938.
13. Arbabi-Ghahroudi M. Camelid Single-Domain Antibodies: Historical Perspective and Future Outlook. Front Immunol. 2017; 8:1589. Epub 2017/12/07. doi: https://doi.org/10.3389/fimmu.2017.01589. PubMed PMID: 29209322; PubMed Central PMCID: PMCPMC5701970.
14. Liu J L, Shriver-Lake L C, Anderson G P, Zabetakis D, Goldman E R. Selection, characterization, and thermal stabilization of llama single domain antibodies towards Ebola virus glycoprotein. Microb Cell Fact. 2017; 16(1):223. Epub 2017/12/14. doi: https://doi.org/10.1186/s12934-017-0837-z. PubMed PMID: 29233140; PubMed Central PMCID: PMCPMC5726015.
15. Goldman E R, Anderson G P, Conway J, Sherwood L J, Fech M, Vo B, et al. Thermostable llama single domain antibodies for detection of botulinum A neurotoxin complex. Anal Chem. 2008; 80(22):8583-91. Epub 2008/10/25. doi: https://doi.org/10.1021/ac8014774. PubMed PMID: 18947189; PubMed Central PMCID: PMCPMC2829253.
16. Wu Y, Jiang S, Ying T. Single-Domain Antibodies As Therapeutics against Human Viral Diseases. Front Immunol. 2017; 8:1802. Epub 2018/01/13. doi: https://doi.org/10.3389/fimmu.2017.01802. PubMed PMID: 29326699; PubMed Central PMCID: PMCPMC5733491.
17. Wrapp D, De Vlieger D, Corbett K S, Torres G M, Wang N, Van Breedam W, et al. Structural Basis for Potent Neutralization of Betacoronaviruses by Single-Domain Camelid Antibodies. Cell. 2020; 181(5):1004-15 e15. Epub 2020/05/07. doi: https://doi.org/10.1016/j.cell.2020.04.031. PubMed PMID: 32375025; PubMed Central PMCID: PMCPMC7199733.
18. Hanke L, Vidakovics P, Sheward D J, Das H, Schulte T, Morro A M, et al. An alpaca nanobody neutralizes SARS-CoV-2 by blocking receptor interaction. bioRxiv. 2020. Epub 06/02/2020. doi: https://doi.org/10.1101/2020.06.02.130161.
19. Ou J, Zhou Z, Dai R, Zhang J, Lan W, Zhao S, et al. Emergence of RBD mutations in circulating SARS-CoV-2 strains enhancing the structural stability and human ACE2 receptor affinity of the spike protein. bioRxiv 2020. Epub 04/20/2020. doi: https://doi.org/10.1101/2020.03.15.991844.
20. Dong J, Huang B, Jia Z, Wang B, Gallolu Kankanamalage S, Titong A, et al. Development of multi-specific humanized llama antibodies blocking SARS-CoV-2/ACE2 interaction with high affinity and avidity. Emerg Microbes Infect. 2020; 9(1):1034-6. Epub 2020/05/15. doi: https://doi.org/10.1080/22221751.2020.1768806. PubMed PMID: 32403995.
21. Detalle L, Stohr T, Palomo C, Piedra PA, Gilbert B E, Mas V, et al. Generation and Characterization of ALX-0171, a Potent Novel Therapeutic Nanobody for the Treatment of Respiratory Syncytial Virus Infection. Antimicrob Agents Chemother. 2016; 60(1):6-13. Epub 2015/10/07. doi: https://doi.org/10.1128/AAC.01802-15. PubMed PMID: 26438495; PubMed Central PMCID: PMCPMC4704182.
22. Ulrichts H, Silence K, Schoolmeester A, de Jaegere P, Rossenu S, Roodt J, et al. Antithrombotic drug candidate ALX-0081 shows superior preclinical efficacy and safety compared with currently marketed antiplatelet drugs. Blood. 2011; 118(3):757-65. Epub 2011/05/18. doi: https://doi.org/10.1182/blood-2010-11-317859. PubMed PMID: 21576702.
23. Van Heeke G, Allosery K, De Brabandere V, De Smedt T, Detalle L, de Fougerolles A. Nanobodies® as inhaled biotherapeutics for lung diseases. Pharmacol Ther. 2017; 169:47-56. Epub 2016/07/05. doi: https://doi.org/10.1016/j.pharmthera.2016.06.012. PubMed PMID: 27373507.
24. Larios Mora A, Detalle L, Gallup J M, Van Geelen A, Stohr T, Duprez L, et al. Delivery of ALX-0171 by inhalation greatly reduces respiratory syncytial virus disease in newborn lambs. MAbs. 2018; 10(5):778-95. Epub 2018/05/08. doi: https://doi.org/10.1080/19420862.2018.1470727. PubMed PMID: 29733750; PubMed Central PMCID: PMCPMC6150622.
25. Imai M, Iwatsuki-Horimoto K, Hatta M, Loeber S, Halfmann P J, Nakajima N, et al. Syrian hamsters as a small animal model for SARS-CoV-2 infection and countermeasure development. Proc Natl Acad Sci USA. 2020. Epub 2020/06/24. doi: https://doi.org/10.1073/pnas.2009799117. PubMed PMID: 32571934.
26. McCray P B, Jr., Pewe L, Wohlford-Lenane C, Hickey M, Manzel L, Shi L, et al. Lethal infection of K18-hACE2 mice infected with severe acute respiratory syndrome coronavirus. J Virol. 2007; 81(2):813-21. Epub 2006/11/03. doi: https://doi.org/10.1128/JVI.02012-06. PubMed PMID: 17079315; PubMed Central PMCID: PMCPMC1797474.
27. Singh D K, Ganatra S R, Singh B, Cole J, Alfson K J, Clemmons E, et al. SARS-CoV-2 infection leads to acute infection with dynamic cellular and inflammatory flux in the lung that varies across nonhuman primate species. bioRxiv. 2020. Epub 06/05/2020. doi: https://doi.org/10.1101/2020.06.05.136481.
28. Pardon E, Laeremans T, Triest S, Rasmussen S G, Wohlkonig A, Ruf A, et al. A general protocol for the generation of Nanobodies for structural biology. Nat Protoc. 2014; 9(3):674-93. Epub 2014/03/01. doi: https://doi.org/10.1038/nprot.2014.039. PubMed PMID: 24577359; PubMed Central PMCID: PMCPMC4297639.
29. Sievers F, Wilm A, Dineen D, Gibson T J, Karplus K, Li W, et al. Fast, scalable generation of high-quality protein multiple sequence alignments using Clustal Omega. Mol Syst Biol. 2011; 7:539. Epub 2011/10/13. doi: https://doi.org/10.1038/msb.2011.75. PubMed PMID: 21988835; PubMed Central PMCID: PMCPMC3261699.

TABLE 3
Sequences disclosed herein

| SEQ | | |
|---|---|---|
| ID | | |
| NO: | AMINO ACID OR NUCLEOTIDE SEQUENCE | |
| 1 | DVQLQESGGD LVQPGGSLRL SCAASGFTLD YYAIGWFRQA PGKEREGVSC ISSSDGSTYY | 60 |
| | ADSVKGRFTS SRDNAKNTVY LQMNSLKPED TAVYYCAAVP STYYNGSYYY TCHPGGMDYW | 120 |
| | GKGTQVTVSS | 130 |
| 2 | DVQLQESGGG LVQPGGSLRL SCAVSGFTLD YYAIGWFRQA PGKEREGVSC ISSSDGSTYY | 60 |
| | ADSVKGRFTS SRDNAKNTVY LQMNSLKPED TAVYYCAAVP STYYSGTYYY NCHPGGMDYW | 120 |
| | GKGTQVTVSS | 130 |
| 3 | DVQLQESGGG LVQPGGSLRL SCAASGLTLD YYTIGWFRQA PGKEREGVSC ISSSDDSTYY | 60 |
| | ADSVKGRFTI SRDNAKNTVY LQMNSLKPED TAVYYCATAP GTYYKGSYYP MCHYYGMDYW | 120 |
| | GKGTQVTVSS | 130 |
| 4 | DVQLQESGGG LVQPGGSLRL SCAVSGFTLD YYAIGWFRQA PGKEREGVAC ISSSDGTTYY | 60 |
| | ADSVKGRFTI SRDNAKNTVY LQMNSLKPED TAVYYCATRP LTYYSGSYYT TCSDYGMDYW | 120 |
| | GKGTLVTVSS | 130 |
| 5 | DVQLQESGGG LVQPGGSLRL SCAASGFTLD YYAIGWFRQA PGKEREGVSC ISNSDGSTYY | 60 |
| | ADSVKGRFTT SRDNAKNTVY LQMNSLKPED TAVYYCAAVP STYYSGSYYY TCHPGGMDYW | 120 |
| | GKGTQVTVSS | 130 |
| 6 | DVQLQESGGG LVQSGGSLRL SCAASGFTLD YYAIGWFRQA PGKEREGVSC ISNSDGSTYY | 60 |
| | ADSVKGRFTT SRDNAKNTVY LQMNSLKPED TAVYYCAAVP STYYSGSYYY TCHPGGMDYW | 120 |
| | GKGTQVTVSS | 130 |
| 7 | DVQLQESGGG LVQPGGSLRL SCAASGFTLD YYAIGWFRQA PGKEREGVSC ISNSDGSTYY | 60 |
| | ADSVKGRFTT SRDNAKNTVY LQMNSLKPED TAVYYCAAVP STYYSGSYYY TCHPGGMDYW | 120 |
| | GKGTLVTVSS | 130 |
| 8 | DVQLQESGGG LVQPGGSLRL SCAASGFTLD YYAIGWFRQA PGKEREGVSC ISNSGGSTYY | 60 |
| | ADSVKGRFTT SRDNAKNTVY LQMNSLKPED TAVYYCAAVP STYYSGSYYY TCHPGGMDYW | 120 |
| | GKGTQVTVSS | 130 |
| 9 | DVQLQESGGG LVQSGGSLRL SCAASGFTLD YYAIGWFRQA PGKEREGVSC ITNSDGSTYY | 60 |
| | ADSVKGRFTT SRDNAKNTVY LQMNSLKPED TAVYYCASFP STYYSGSYYY TCHPGGMDYW | 120 |
| | GKGTQVTVSS | 130 |
| 10 | DVQLQESGGG LVQPGGSLRL SCAASGFTLD YYAIGWFRQA PGKEREGVSC ISSSDGSTYY | 60 |
| | ADSVKGRFTI SRDNAKNTVY LQMNSLKPDD TAVYYCAAAL SEGGYTIDGS SWCYHSVYGM | 120 |
| | DYWGKGTQVT VSS | 133 |
| 11 | DVQLQESGGG SVEAGGSLRL SCAASGVTLD YYAIGWFRQA PGKEREGVSC ISSSDGSTYY | 60 |
| | ADSVKGRFTT SRDNAKNTVY LQMNSLKPED TADYYCAAVP STYYSGTYYY NCHPGAMHYW | 120 |
| | GKGTQVTVSS | 130 |
| 12 | DVQLQESGGG LVQPGGSLRL SCAASGLTLD YYAIGWFRQA PGKEREGVSC ISSSDGSTYY | 60 |
| | ADSVKGRFTT SRDNAKNTVY LQMNSLKPED TAVYYCAAVP STYYSGTYYY TCHPGGMDYW | 120 |
| | GKGTQVTVSS | 130 |
| 13 | DVQLQESGGG LVQPGGSLRL SCAASGLTLD YYAIGWFRQA PGKEREGVSC ISSSDGSTYY | 60 |
| | ADSVKGRFTT SRDNAKNTVY LQMNSLKPED TAVYYCAAVP STYYSGTYYY TCHPGGMDYW | 120 |
| | GKGTLVTVSS | 130 |
| 14 | TLDYYAIG | |
| 15 | TLDYYTIG | |
| 16 | CISSSDGSTY | |
| 17 | CISSSDDSTY | |
| 18 | CISSSDGTTY | |
| 19 | CISNSDGSTY | |
| 20 | CISNSGGSTY | |
| 21 | CITNSDGSTY | |
| 22 | VPSTYYNGSYYYTCHPGGMD | |
| 23 | VPSTYYSGTYYYNCHPGGMD | |
| 24 | APGTYYKGSYYPMCHYYGMD | |
| 25 | RPLTYYSGSYYTTCSDYGMD | |
| 26 | VPSTYYSGSYYYTCHPGGMD | |
| 27 | FPSTYYSGSYYYTCHPGGMD | |
| 28 | ALSEGGYTIDGSSWCYHSVYGMD | |
| 29 | VPSTYYSGTYYYNCHPGAMH | |
| 30 | VPSTYYSGTYYYTCHPGGMD | |
| 31 | MAQVQLVETG GGLVQPGGSL RLSCAASGFT FSSVYMNWVR QAPGKGPEWV SRISPNSGNI | 60 |
| | GYTDSVKGRF TISRDNAKNT LYLQMNNLKP EDTALYYCAI GLNLSSSSVR GQGTQVTSS | 119 |
| 32 | QVQLQESGGG LVQAGGSLRL SCAASGRTFS EYAMGWFRQA PGKEREFVAT ISWSGGSTYY | 60 |
| | TDSVKGRFTI SRDNAKNTVY LQMNSLKPDD TAVYYCAAAG LGTWSEWDY DYDYWGQGTQ | 120 |
| | VTVSS | 125 |
| 33 | RVQPTESIVR FPNITNLCPF GEVFNATRFA SVYAWNRKRI SNCVADYSVL YNSASFSTFK | 60 |
| | CYGVSPTKLN DLCFTNVYAD SFVIRGDEVR QIAPGQTGKI ADYNYKLPDD FTGCVIAWNS | 120 |
| | NNLDSKVGGN YNYLYRLFRK SNLKPFERDI STEIYQAGST PCNGVEGFNC YFPLQSYGFQ | 180 |
| | PTNGVGYQPY RVVVLSFELL HAPATVCGPK KSTNLVKNKC VNF22 3 | |
| 34 | gtcctggctgctcttctacaagg | |
| 35 | ggtacgtgctgttgaactgttcc | |
| 36 | ccggccatggctgatgtgcagctgcaggagtctggrggag g | |
| 37 | gtgcggccgctgaggagacggtgacctgggt | |
| 38 | cgtatgttgtgtggaattgtgagc | |
| 39 | tatctctcgagaaaagagatgtgcagctgcaggagtctg | |
| 40 | ttgttctagattagtgatggtgatgatgatgtgcggccgc | |

1. A VHH domain that specifically binds a severe acute respiratory syndrome coronavirus spike protein receptor binding domain, wherein the amino acid sequence of the VHH domain comprises any one of SEQ ID NOs:1-13.

2. The VHH domain of claim 1, wherein the amino acid sequence of the VHH domain comprises SEQ ID NO:12.

3. A VHH domain that specifically binds a severe acute respiratory syndrome coronavirus spike protein receptor binding domain, wherein the VHH domain comprises a CDR1 as set forth in SEQ ID NOs:14 or 15; CDR 2 as set forth in any one of SEQ ID NOs:16, 17, 18, 19, 20, or 21; and CDR3 as set forth in any one of SEQ ID NOs:23-30.

4. A VHH domain that specifically binds a severe acute respiratory syndrome coronavirus spike protein receptor binding domain, wherein the VHH domain comprises:
(a) a CDR1 as set forth in SEQ ID NO:14, a CDR2 as set forth in SEQ ID NO:16, and CDR3 as set forth in SEQ ID NO:22;
(b) a CDR1 as set forth in SEQ ID NO:14, a CDR2 as set forth in SEQ ID NO:16, and a CDR3 as set forth in SEQ ID NO:23;
(c) a CDR1 as set forth in SEQ ID NO:15, a CDR2 as set forth in SEQ ID NO:17, and a CDR3 as set forth in SEQ ID NO:24;
(d) a CDR1 as set forth in SEQ ID NO:14, a CDR2 as set forth in SEQ ID NO:18, and a CDR3 as set forth in SEQ ID NO:25;
(e) a CDR1 as set forth in SEQ ID NO:14, a CDR2 as set forth in SEQ ID NO:19, and a CDR3 as set forth in SEQ ID NO:26;
(f) a CDR1 as set forth in SEQ ID NO:14, a CDR2 as set forth in SEQ ID NO:20, and a CDR3 as set forth in SEQ ID NO:26;
(g) a CDR1 as set forth in SEQ ID NO:14, a CDR2 as set forth in SEQ ID NO:21, and a CDR3 as set forth in SEQ ID NO:27;
(h) a CDR1 as set forth in SEQ ID NO:14, a CDR2 as set forth in SEQ ID NO:16, and a CDR3 as set forth in SEQ ID NO:28;
(i) a CDR1 as set forth in SEQ ID NO:14, a CDR2 as set forth in SEQ ID NO:16, and a CDR3 as set forth in SEQ ID NO:29; or
(j) a CDR1 as set forth in SEQ ID NO:14, a CDR2 as set forth in SEQ ID NO:16, and a CDR3 as set forth in SEQ ID NO:30.

5. The VHH domain of claim 1, wherein
the amino acid sequence of the severe acute respiratory syndrome coronavirus spike protein receptor binding domain comprises SEQ ID NO:33.

6. The VHH domain of claim 1, wherein
the severe acute respiratory syndrome coronavirus spike protein binding domain is a severe acute respiratory syndrome coronavirus 2 (SARS-CoV-2) spike protein binding domain.

7. The VHH domain of claim 6, wherein the VHH domain has a binding affinity of at least 3 nM.

8. (canceled)

9. A nucleic acid, comprising a nucleotide sequence encoding the amino acid sequence of the VHH domain of claim 1.

10-11. (canceled)

12. An immunoconjugate, comprising (a) the VHH domain of claim 1 and (b) a conjugating part selected from a detectable moiety, a drug, a toxin, or a cytokine.

13. The immunoconjugate of claim 12, wherein the detectable moiety is selected from fluorophores, immuno-histochemical tracers, positron emission tomography (PET) tracers, near infrared spectrometer (NIR) probes, single-photon emission computerized tomography (SPECT), magnetic particle imaging, magnetic resonance imaging contrast agents, ultrasound contrast agents, and radio-isotopes.

14. A pharmaceutical composition, comprising:
(a) a therapeutically effective amount of the VHH domain of claim 1; and
(b) a pharmaceutically acceptable carrier.

15. A method of treating severe acute respiratory syndrome coronavirus, the method comprising administering to a subject a therapeutically effective amount of the pharmaceutical composition of claim 14.

16. A method of preventing severe acute respiratory syndrome coronavirus, the method comprising administering to a subject a therapeutically effective amount of the pharmaceutical composition of claim 14.

17. A method of ameliorating severe acute respiratory syndrome coronavirus, the method comprising administering to a subject a therapeutically effective amount of the pharmaceutical composition of claim 14.

18. The method of claim 15, wherein the severe acute respiratory syndrome coronavirus is severe acute respiratory syndrome coronavirus 2 (SARS-CoV-2).

19. The method of claim 15, wherein the VHH domain is administered parenterally.

20. (canceled)

21. A method of diagnosis of severe acute respiratory syndrome coronavirus in a subject using the VHH domain of claim 1.

22. (canceled)

23. A method for diagnosing a severe acute respiratory syndrome coronavirus infection in a patient, comprising detecting in a sample from the patient a spike protein receptor binding domain from a severe acute respiratory syndrome coronavirus, wherein the sample is contacted with the VHH domain of claim 1.

24. A method for detecting a severe acute respiratory syndrome coronavirus on a contaminated surface, comprising detecting in a sample from the contaminated surface a spike protein receptor binding domain from a severe acute respiratory syndrome coronavirus wherein the sample is contacted with the VHH domain of claim 1.

25. A method of decontaminating a severe acute respiratory syndrome coronavirus contaminated surface, comprising: contacting the surface which is contaminated with a composition comprising the VHH domain of claim 1 for sufficient time to substantially reduce the virus on the surface.

26. The method of claim 21, wherein the severe acute respiratory syndrome coronavirus is severe acute respiratory syndrome coronavirus 2 (SARS-CoV-2).

27. A binding polypeptide complex, comprising a dimer of a first VHH domain and a second VHH domain, wherein the first VHH domain and the second VHH domain comprise the VHH domain of claim 1, wherein the first VHH and the second VHH domains are linked by a dimerization domain.
